(19) United States
(12) Patent Application Publication
(10) Pub. No.: US 2024/0058217 A1
(43) Pub. Date: Feb. 22, 2024

(54) PIERCING CONNECTIONS FOR PRE-FILLED PLASTIC VIALS

(71) Applicants: Jeff Price, Windermere, FL (US); Koska Family Limited, East Sussex (GB)

(72) Inventors: Jeff Price, Windermere, FL (US); Marc Andrew Koska, Dartmouth (GB)

(21) Appl. No.: 18/267,365

(22) PCT Filed: Dec. 17, 2021
(86) PCT No.: PCT/US2021/064155

§ 371 Date: Jun. 14, 2023

Related U.S. Application Data
(60) Provisional application No. 63/127,238, filed on Dec. 18, 2020.

(51) Int. Cl. A61J 1/14; A61J 1/20; A61J 1/06

(52) U.S. Cl. CPC A61J 1/1406; A61J 1/1481; A61J 1/2096; A61J 1/2089; A61J 1/201; A61J 1/065

(57) ABSTRACT

A pre-filled blow-fill-seal (BFS) vial can have a fluid seal at one end thereof that can be breached by a piercing element within a connector when a neck of the BFS vial is fully inserted into the connector. The piercing element can comprise or define one or more fluid conduits that allow fluid to flow from an interior of the BFS vial to an outlet port of the connector, thereby allowing the contents to be dispensed from the BFS vial. The connector can facilitate coupling of the BFS vial for subsequent administration of the contents therein to a patient. For example, the outlet port of the connector can be coupled to an administration assembly, such as a syringe, nozzle, intravenous (IV) therapy device, or another device.

### CROSS-REFERENCE TO RELATED APPLICATIONS

Benefit and priority is claimed to, and this is a National Stage pursuant to 35 U.S.C. § 371 of, International Patent Application No. PCT/US21/064155, having an international filing date of Dec. 17, 2021 and titled “Piercing Connectors for Pre-Filled Plastic Vials”, which International Patent Application claims benefit of and priority under 35 U.S.C. § 119(e) to, and is a Non-provisional of U.S. Provisional Patent Application No. 63/127,238, filed on Dec. 18, 2020, and titled “Piercing Connectors for Pre-filled Plastic Vials.” Each of these patent applications is hereby incorporated by reference herein in its entirety and for all purposes.

### BACKGROUND

Every year, millions of people become infected and die from a variety of diseases, some of which are vaccine-preventable. Although vaccination has led to a dramatic decline in the number of cases of several infectious diseases, some of these diseases remain quite common. In many instances, large populations of the world, particularly in developing countries, suffer from the spread of vaccine-preventable diseases due to ineffective immunization programs, either because of poor implementation, lack of affordable vaccines, or inadequate devices for administering vaccines, or combinations thereof.

Some implementations of immunization programs include administration of vaccines via a reusable syringe. However, in many situations, particularly in developing countries, the administration of vaccines occur outside of a hospital and may be provided by a non-professional, such that injections are given to patients without carefully controlling access to syringes. The use of reusable syringes under those circumstances increases the risk of infection and spread of blood-borne diseases, particularly when syringes, which have been previously used and are no longer sterile, are used to administer subsequent injections. For example, the World Health Organization (WHO) estimates that blood-borne diseases, such as Hepatitis and human immunodeficiency virus (HIV), are being transmitted due to reuse of such syringes, resulting in the death of more than one million people each year.

Previous attempts at providing single-use or disposable injection devices to remedy such problems in the industry have achieved measurable success but have failed to adequately remedy the existing problems. Pre-filled, single-use injection devices manufactured via injection molding or Form-Fill-Seal (FFS) processes, such as the Uniject™ device available from the Becton, Dickinson and Company of Franklin Lakes, NJ, for example, while offering precise manufacturing tolerances in the range of two thousandths of an inch (0.002-in; 50.8 μm) to four thousandths of an inch (0.004-in; 101.6 μm)—for hole diameters in molded parts, require separate sterilization processes (e.g., gamma radiation) that are not compatible with certain fluids, provide production rates limited to approximately nine thousand (9,000) non-sterile units per hour, and can be provided to an end-user for approximately one dollar and forty cents ($1.40) per dose/unit.

Utilization of Blow-Fill-Seal (BFS) processes to create injectable devices has generally been limited to processes that insert a cannula (needle) during the manufacturing process such that it is integral to the BFS vial. Such efforts have not been successful for various reasons, not the least of which is the increased cost and increased manufacturing delay created by the cannula insertion process. Modern attempts to utilize BFS vials as injectable delivery mechanisms generally rely on manual removal of a sealing tab or stem from the BFS vial that tears the BFS vial to expose a fluid contained therein.

### SUMMARY

Embodiments of the disclosed subject matter provide systems, assemblies, and methods for piercing and/or accessing sealed medical delivery assemblies, such as pre-filled blow-fill-seal (BFS) vials. In some embodiments, the BFS vial can have a fluid seal at one end thereof that can be breached by a piercing element within a connector when a neck of the BFS vial is fully inserted into the connector. In some embodiments, the piercing element can comprise or define one or more fluid conduits that allow fluid to flow from an interior of the BFS vial to an outlet port of the connector, thereby allowing the contents to be dispensed from the BFS vial. In some embodiments, the connector can facilitate coupling of the BFS vial for subsequent administration of the contents therein to a patient. For example, the outlet port of the connector can be coupled to an administration assembly, such as a syringe, nozzle, intravenous (IV) therapy device, or another device.

In one or more embodiments, a pre-filled medical delivery assembly can comprise a blow-fill-seal (BFS) vial and a connector. The BFS vial can have first and second longitudinal ends. The BFS vial can comprise at least one reservoir having a fluid therein and a longitudinally-extending neck portion having a fluid seal at the first longitudinal end of the BFS vial. The connector can comprise an inlet port, an outlet port, and a piercing element. The inlet port can connect to a first interior volume, and the outlet port can connect to a second interior volume in fluid communication with the first interior volume. The piercing element can have a least a piercing tip disposed within the first interior volume. The neck portion of the BFS vial can extend through the inlet port into the first interior volume, such that at least part of the piercing element breaches and extends through the fluid seal.

Any of the various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosed technology will become more apparent from the following detailed description, which proceeds with reference to the accompanying figures.

### BRIEF DESCRIPTION OF THE DRAWINGS

Where applicable, some elements may be simplified or otherwise not illustrated in order to assist in the illustration and description of underlying features. For example, in some figures, some components have been illustrated using a partial or cutaway view in order to illustrate internal interaction of components. Throughout the figures, like reference numerals denote like elements. An understanding of embodiments described herein and many of the attendant advantages thereof may be readily obtained by reference to the following detailed description when considered with the accompanying drawings, wherein:

FIGS. 1A-1B are side and front views, respectively, of an exemplary pre-filled blow-fill-seal (BFS) vial for use in a medical delivery assembly, according to one or more embodiments of the disclosed subject matter;

FIGS. 1C-1D are side and front views, respectively, of another exemplary pre-filled BFS vial for use in a medical delivery assembly, according to one or more embodiments of the disclosed subject matter;

FIGS. 2A-2C show side, perspective, and front views, respectively, of an exemplary medical delivery assembly, according to one or more embodiments of the disclosed subject matter;

FIGS. 3A-3B are side cross-sectional views of a generalized piercing connector and BFS vial in sealed and pierced configurations, respectively, according to one or more embodiments of the disclosed subject matter;

FIGS. 4A-4D are top, side, bottom, and perspective views, respectively, of a first exemplary piercing connector, according to one or more embodiments of the disclosed subject matter;

FIGS. 4E-4F are sectional perspective and cross-sectional views, respectively, of the first exemplary piercing connector of FIGS. 4A-4D;

FIG. 4G is a detailed cross-sectional view of a piercing element of the first exemplary piercing connector of FIGS. 4A-4F;

FIGS. 5A-5D are top, side, bottom, and perspective views, respectively, of a second exemplary piercing connector, according to one or more embodiments of the disclosed subject matter;

FIGS. 5E-5G are sectional perspective and cross-sectional views, respectively of the second exemplary piercing connector of FIGS. 5A-5D;

FIG. 5H is a detailed cross-sectional view of a piercing element of the second exemplary piercing connector of FIGS. 5A-5G;

FIGS. 6A-6B are side views of an exemplary medical delivery assembly with a third exemplary piercing connector in unassembled and assembled configurations, respectively, according to one or more embodiments of the disclosed subject matter;

FIGS. 6C-6D are sectional perspective and cross-sectional views, respectively, of the exemplary medical delivery assembly of FIGS. 6A-6B with access cap removed;

FIG. 6E is a sectional perspective view of the third exemplary piercing connector of FIGS. 6A-6D;

FIG. 6F is a sectional perspective view of the exemplary medical delivery assembly of FIGS. 6A-6D coupled to an exemplary delivery device, according to one or more embodiments of the disclosed subject matter;

FIG. 7A is a cross-sectional view of an exemplary medical delivery assembly with a fourth exemplary piercing connector, according to one or more embodiments of the disclosed subject matter; and

FIG. 7B is a cross-sectional view of the exemplary medical delivery assembly of FIG. 7A coupled to an exemplary delivery device, according to one or more embodiments of the disclosed subject matter.

### DETAILED DESCRIPTION

### I. Introduction

Described herein are systems, assemblies, and methods for piercing systems and connectors for accessing sealed medical delivery assemblies that can overcome drawbacks of current delivery devices and methods. For example, pre-filled and/or dual-chamber medical delivery assemblies of some embodiments may include a Blow-Fill-Seal (BFS) vial or bottle that can be coupled to a specialized collar or connector that facilitates coupling of an administration member (e.g., a needle) to the BFS vial. In some embodiments, such a pre-filled medical delivery assembly may be selectively actuated by application of mating force that urges the piercing connector to axially advance and pierce a fluid reservoir of the BFS vial. In some embodiments, a specialized BFS coupling, connector, and/or collar may be snapped onto a BFS vial, thereby puncturing the BFS vial. Utilization of such systems that employ BFS vials and connectors configured to selectively pierce the BFS vials may be advantageous and may address various shortcomings of previous systems.

In some embodiments, BFS vials (e.g., due to the nature of the BFS manufacturing process) may not require separate sterilization (and thereby may be compatible with a wider array of liquid agents), may provide enhanced production rates of sterile/aseptic units per hour, and/or may be provided to an end-user for significantly lower per dose/unit costs. In some embodiments, these advantages may come with an attendant drawbacks of reduced manufacturing tolerances and other disadvantages of utilizing a “soft” plastic (e.g., having a Shore/Durometer “D” hardness of between 60 and 70). BFS processes may, for example, offer less precise manufacturing tolerances in the range of five hundredths of an inch (0.05-in; 1.27 mm) to fifteen hundredths of an inch (0.15-in; 3.81 mm)—for linear dimensions, e.g., in accordance with the standard ISO 2768-1 “General tolerances for linear and angular dimensions without individual tolerance indications” published by the International Organization for Standardization (ISO) of Geneva, Switzerland (Nov. 15, 1989), which is incorporated herein by reference.

### II. BFS Vials

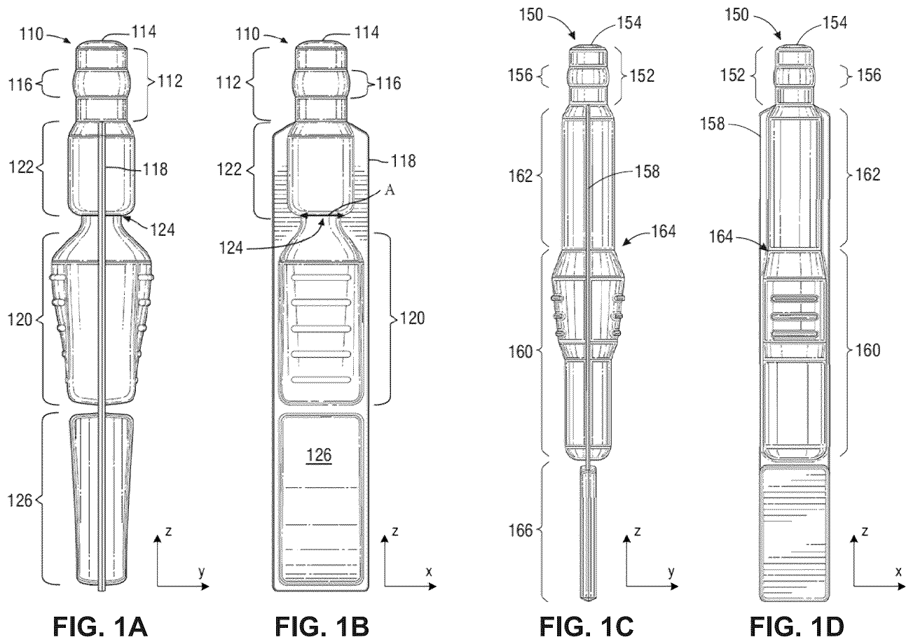

Referring initially to FIGS. 1A-1B, various views of an exemplary BFS vial 110 are shown. The BFS vial 110 (also referred to herein as a BFS bottle, container, or module) can comprise and/or define a vial neck 112, a fluid seal 114, a mounting portion 116 (also referred to herein as a coupling portion, collar, or flange), and/or one or more reservoirs 120, 122. In some embodiments, the BFS vial 110 may also comprise a body flange 118 (also referred to herein as a bottle flange or interconnecting web), which may, for example, comprise unmolded portions of fused parison. In some embodiments, the body flange 118 connects the BFS vial 110 to adjacent vials simultaneously formed by a BFS manufacturing process.

In some embodiments, the BFS vial 110 may also comprise and/or define a tab 126 upon and/or in which various identifying information (e.g., descriptive of the fluid agent contained within the vial) is provided. For example, the identifying information may comprise various printed, engraved (e.g., laser engraved), embossed, adhered, radio-frequency identification (RFID), quick response (QR) code, barcode, and/or other informational objects (human and/or computer-readable) that are indicative of one or more characteristics of the vial, a product card of which the vial was a part, the manufacturing process/device/location, and/or the fluid agent(s). In some embodiments, the identifying information may comprise first information disposed on a first side or face of the tabs 126 (e.g., the front side as shown in FIG. 1A) and/or second information disposed on a second side or face of the tabs 126 (not shown in FIG. 1A).

In the illustrated example of FIGS. 1A-1B, the BFS vial 110 may comprise and/or define a neck 112 that terminates at fluid seal 114 disposed at a first end of the vial 110. For example, the fluid seal 114 may comprise a portion of the molded BFS vial 110 that is configured to be pierced to expel the fluid, e.g., such as by providing a flat or planar piercing surface and/or by being oriented normal to an axis (e.g., longitudinal direction, z) of the BFS vial 110 (and/or the pre-filled medical delivery assembly 100). In some embodiments, the fluid seal 114 may comprise a foil, wax, paper, and/or other thin, pierceable object or layer coupled to the BFS vial 110.

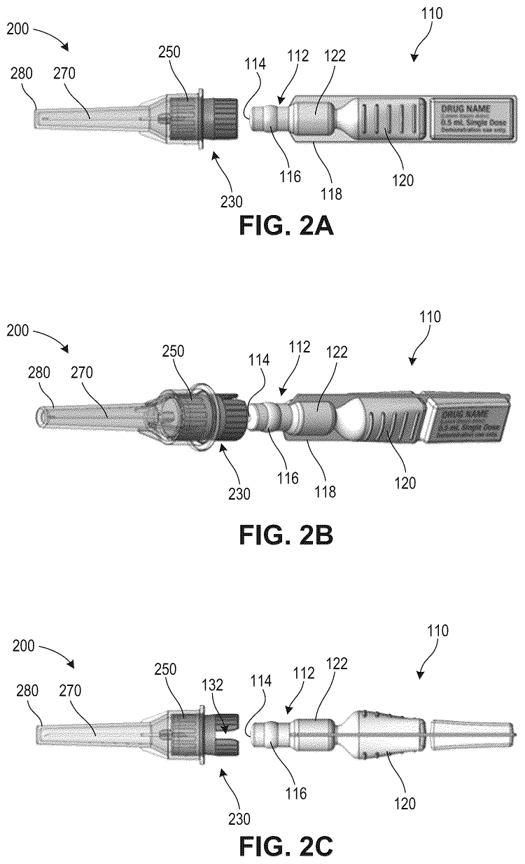

In some embodiments, the neck 112 may comprise and/or define mounting portion 116. For example, the mounting portion 116 can be formed as an axially-elongated and/or rounded exterior flange or projection, e.g., the “doughnut”-shaped or toroidal-shaped exterior flange or collar, as depicted. Alternatively or additionally, the mounting portion can comprise a detent, projection, protrusion, tab, notch, etc. The mounting portion 116 may, for example, provide a radially-elastic mating surface that is operable to provide a selective engagement or fit within a hub of an administration component or assembly (e.g., the piercing element of any of FIGS. 2A-7B).

In some embodiments, the one or more reservoirs 120, 122 may be filled (fully or partially) with a fluid or other agent (not separately shown) to be delivered, e.g., to a patient (not shown). According to some embodiments, the fluid may be injected into the BFS vial 110 during manufacture via a BFS process (e.g., in a sterile environment) and sealed within the BFS vial 110 via the fluid seal 114. In some embodiments, the reservoirs 120, 122 may be joined by a constriction 124 (also referred to herein as a passage, juncture, junction, depression, indentation, relief, geometric transition, grip point, valve, restriction, or narrowed portion), as shown in FIGS. 1A-1B. According to some embodiments, the constriction 124 can restrict flow such that the fluid may readily enter one of the dispensing reservoirs 122 and the collapsible reservoir 120, but may not readily return to the other reservoir 120, 122. Such a constriction may in some embodiments, provided advantages as described in International Publication No. WO 2021/207040, published Oct. 14, 2021 and titled “Systems and Methods for Pre-filled Medical Delivery,” which is incorporated by reference herein in its entirety. Alternatively, in some embodiments, the BFS vial can be provided with a single reservoir or multiple reservoirs without a corresponding constriction between the reservoirs. For example, FIGS. 1C-1D illustrate such a BFS vial 150 comprising and/or defining a vial neck 152, a fluid seal 154, a coupling portion 156, a body flange 158, a first reservoir 160, a second reservoir 162, and a tab 166. As shown in FIGS. 1C-1D, the connection 164 between the first and second reservoirs 160, 162 can be coupled together without any constriction therebetween.

As generally understood, the fluid or drug agent sealed within the BFS vial 110 (or BFS vial 150) may include any type of agent to be injected into a patient (e.g., mammal, either human or non-human, or any other animal) and capable of producing an effect (alone, or in combination with an active ingredient). Accordingly, the agent may include, but is not limited to, a vaccine, a drug, a therapeutic agent, a medicament, a diluent, and/or the like. According to some embodiments, either or both of the fluid agent and the active ingredient (i.e., the drug agent and/or components thereof) may be tracked, monitored, checked for compatibility with each other, etc., such as by utilization of electronic data storage devices (not shown) coupled to the various modules or components of the pre-filled multi-liquid medical delivery assembly, such as the BFS vial 110, housing 250, and/or an administration component.

In some embodiments, fewer or more components 110-166 and/or various configurations of the depicted components 110-166 may be included in the BFS vial without deviating from the scope of embodiments described herein. In some embodiments, the components 110-166 may be similar in configuration and/or functionality to similarly named and/or numbered components as described herein. According to some embodiments, the BFS vial 110 may comprise the mounting portion 116 but not the collapsible reservoir 120, and/or the BFS vial 150 may comprise the mounting portion 156 but not the collapsible reservoir 160. In some embodiments, the BFS vial 11 may comprise the mounting portion 116 but not the dispensing reservoir 122, and/or the BFS vial 150 may comprise the mounting portion 156, but not the dispensing reservoir 162.

### III. Pre-Filled Medical Delivery Assemblies

Referring initially to FIGS. 2A-2C, various partially-exploded views of a pre-filled medical delivery assembly 200 according to one or more embodiments are shown. In some embodiments, the pre-filled medical delivery assembly 200 may comprise various inter-connected and/or modular components, such as a BFS vial 110, a mounting connector 130 (also referred to herein as piercing element, component, or collar), a housing 250 (also referred to herein as a hub) that couples to and/or houses an administration member 270 (also referred to herein as an administration component), and/or a cap 280. According to some embodiments, the pre-filled medical delivery assembly 100 may include a modular design consisting of separately constructed components 110, 230, 250, 270, and 280 cooperatively arranged and coupled to one another.

In some embodiments, the BFS vial 110, mounting collar 230, and/or housing 250 may be coupled, e.g., in the field and/or in situ, to provide an active pre-filled (e.g., injectable) medical delivery assembly 200. For example, in some embodiments, the mounting collar 230 may be axially engaged to couple with the BFS vial 110 via application of a mating axial force. The mounting collar 230 may be urged onto the neck 112 of the BFS vial 110, for example, such that a cooperatively shaped interior groove or seat (not shown) accepts the mounting portion 116, thereby removably coupling the BFS vial 110 and the mounting collar 230. In some embodiments, the interior groove (and/or other interior features) and/or the mounting portion 116 may be shaped such that uncoupling of the BFS vial 110 and the mounting collar 230 is mechanically prohibited. According to some embodiments, the one or more coupling features, such as the mirrored axial slits 132 (also referred to herein as axially-extending slots) shown in FIG. 2A, may engage with the body flange 118 (and/or portions thereof) such that rotation of the mounting collar 230 with respect to the BFS vial 110 is restricted in the case that they are coupled.

In some embodiments, the housing 250 may be coupled to the mounting collar 230 via tabs and/or threads (not shown) and/or may comprise the mounting collar 230 (e.g., they may be molded and/or formed as a single object). The housing 250 may comprise internal threads (not shown) that correspond and cooperate with external threads of the mounting collar 230 such that they may be rotationally and/or removably coupled. According to some embodiments the housing 250 may couple to and/or retain the administration member 270. The administration member 270 may be inserted into and/or through the housing 250, for example, such that it comprises a first or administration end (not separately labeled) extending axially distal from the BFS vial 110 and a second end (not shown) disposed within the housing 250 and/or extending into the mounting collar 230, e.g., in the case that the mounting collar 230 is coupled to the housing 250. In some embodiments, the administration end and/or a distal portion of the administration member 270 may be housed, shrouded, and/or covered by the cap 280. According to some embodiments, the cap 280 may be configured to house the administration member 270 and to removably couple to the housing 250 (e.g., by fitting over an external portion thereof).

According to some embodiments, the mounting collar 230 and housing 250 combination may be utilized to couple and/or mate the administration member 270 with the BFS vial 110 to provide a mechanism via which the administration member 270 may be coupled to the soft plastic BFS vial 110 in a reliable manner. Due to the nature of the BFS plastic and/or process and/or the small form-factor of the BFS vial 110, for example, providing external threads (not shown) directly on the neck 112 would not be a viable option for it would result in an imprecise, unreliable, and/or non-water tight coupling (i.e., the threads would be deformable even if they could be properly manufactured to within the desired tolerances, which itself is not a likely result) between the BFS vial 110 and, e.g., housing 250. Applicant has realized, for example, that “soft” plastics required for the BFS process are not susceptible to machining due to heat deformation of machined features during formation attempts as well as deformation due to mechanical stress during utilization.

In some embodiments, the administration member 270 may comprise a needle (or cannula) for at least one of subcutaneous, intramuscular, intradermal, and intravenous injection of the fluid agent into the patient. For example, the needle can have a length of 0.5 mm to 4 mm, inclusive, or in a range of 4 mm to 15 mm, inclusive, or in a range of 15 mm to 30 mm, inclusive, depending on the desired manner of injection. For ease of explanation and description, the figures and the description herein generally refer to the administration member as a needle. However, it should be noted that, in other embodiments, the administration member 270 may include a nozzle (not shown) configured to control administration of the fluid agent to the patient. The nozzle may include a spray nozzle, for example, configured to facilitate dispersion of the fluid agent into a spray. Accordingly, a housing 250 fitted with a spray nozzle may be particularly useful in the administration of a fluid agent into the nasal passage, for example, or other parts of the body that benefit from a spray application (e.g., ear canal, other orifices). In other embodiments, the nozzle may be configured to facilitate formation of droplets of the fluid agent. Thus, the housing can include a droplet nozzle may be useful in the administration of a fluid agent by way of droplets, such as administration to the eyes, topical administration, and the like.

In some embodiments, advancement of the neck 112 of the BFS vial 110 into the mounting collar 230 through to the seated position may cause a portion of the mounting collar 230 to pierce the fluid seal 114. According to some embodiments, the mounting portion 116 may be configured as the doughnut shape (as depicted) to provide various advantages to the pre-filled medical delivery assembly 200. The axial elongation of the mounting portion 116 may, for example, provide for a smooth, uniform, and/or less forceful mating process that is less likely to deform the soft plastic neck 112 of the BFS vial 110 and/or may provide for a lengthened mating surface that is more likely to prevent leakage of the fluid.

Alternatively, in some embodiments, the neck of the BFS vial can be provided with a coupling portion (or mounting flange) constructed for interfacing with threads in the hub to secure the BFS vial to the collar 230 and/or the administration member 270 (e.g., via housing 250). For example, in place of or in addition to mounting portion 116 in neck 112 of BFS vial 110 (or mounting portion 156 in neck 152 of BFS vial 150), the coupling portion of the BFS vial can define and/or comprise an external thread element (e.g., one or more luer-style thread protrusions). In some embodiments, the external thread element can be constructed as an angled exterior flange designed to fit within a cooperative and/or mirrored angled interior groove or track within the hub.

In some embodiments, the mounting collar 230, the housing 250, and/or the cap 280 may be composed of a medical grade material. In some embodiments, the mounting collar 230, the housing 250, and/or the cap 280 may be composed of a thermoplastic polymer or other relatively hard plastic (e.g., greater than 80 on the Rockwell “M” scale; e.g., Rockwell M 85; and/or greater than 110 on the Rockwell “M” scale; e.g., Rockwell R 115), such as, but not limited to, polypropylene, polybenzimidazole, acrylonitrile butadiene styrene (ABS), polystyrene, polyvinyl chloride, polycarbonate, or the like.

In some embodiments, the pre-filled medical delivery assembly 200 may be advantageously manufactured (in mass quantities) in separate parts or portions, namely, at least the “soft” plastic BFS vial 110 portion (e.g., a “first” piece) and the “hard” plastic mounting collar 230 (e.g., the “second” piece) and the housing 250, administration member 270, and the cap 280 portion (e.g., a “third” piece), with such different plastic parts/portions being selectively coupled to administer a medication to a patient. According to some embodiments, the separate parts or portions may comprise the “soft” plastic BFS vial 110 portion (e.g., a “first” piece) and an integrated “hard” plastic mounting collar 230 and housing 250 coupled to the administration member 270 and the cap 280 (e.g., the “second” piece).

In some embodiments, fewer or more components 110-166, 230-280 and/or various configurations of the depicted components 110-166, 230-280 may be included in the pre-filled medical delivery assembly 200 without deviating from the scope of embodiments described herein. In some embodiments, the components 110-166, 230-280 may be similar in configuration and/or functionality to similarly named and/or numbered components as described herein.

### IV. First Exemplary Piercing Connectors and Assemblies Including Such Connectors

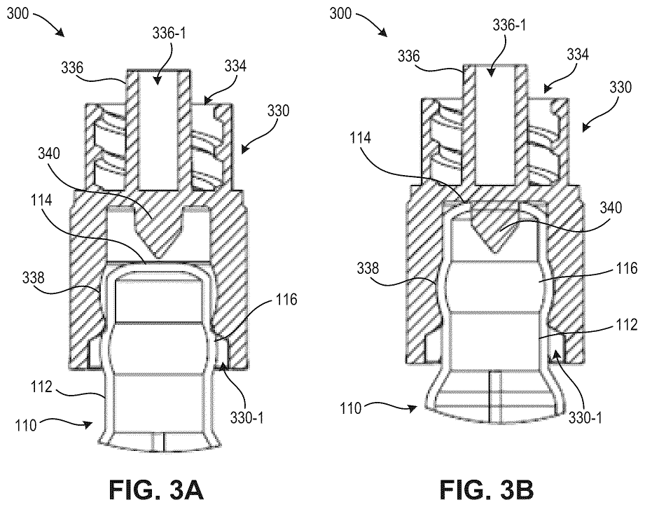

Referring now to FIGS. 3A-3B, an exemplary assembly 300 for piercing pre-filled vials (e.g., BFS vials 110 or 150) according to some embodiments is shown. The assembly 300 may comprise, for example, a BFS vial 110 and a piercing connector 330. The BFS vial 110 can have a neck 112 (e.g., in communication with one or more fluid reservoirs; not shown) that terminates in a fluid seal 114. The neck 112 can comprise and/or define an exterior mounting flange 116 that facilitates coupling of the BFS vial 110 to piercing connector 330. According to some embodiments, the piercing connector 330 may comprise and/or define an inlet opening or port 330-1 disposed at a first or proximal end thereof and sized and/or shaped to accept the neck 112 of the BFS vial 110. In some embodiments, the piercing connector 330 may comprise and/or define, at a second or distal end thereof, threads 334 and/or an outlet conduit 336 (also referred to herein as nozzle, stem, neck, conduit, or lumen) that defines an outlet opening or port 336-1.

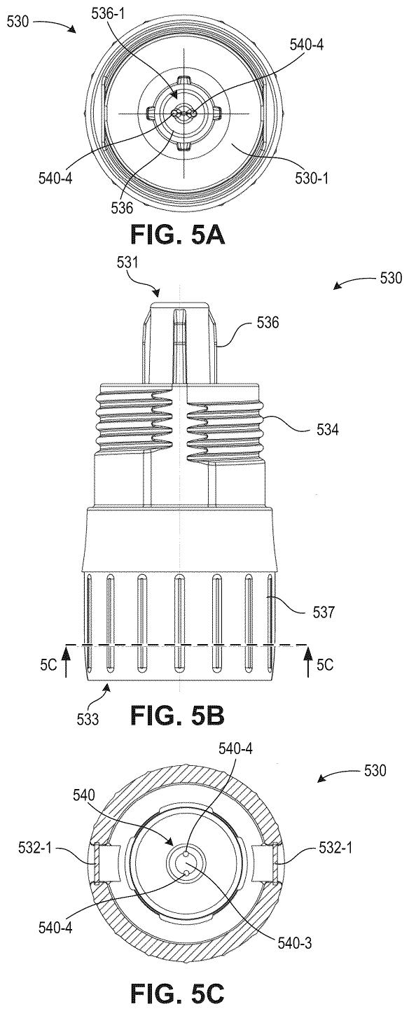

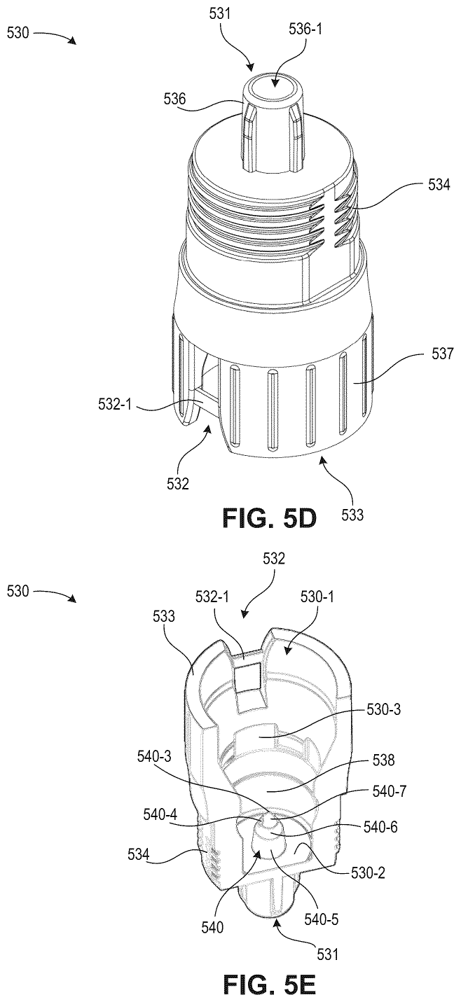

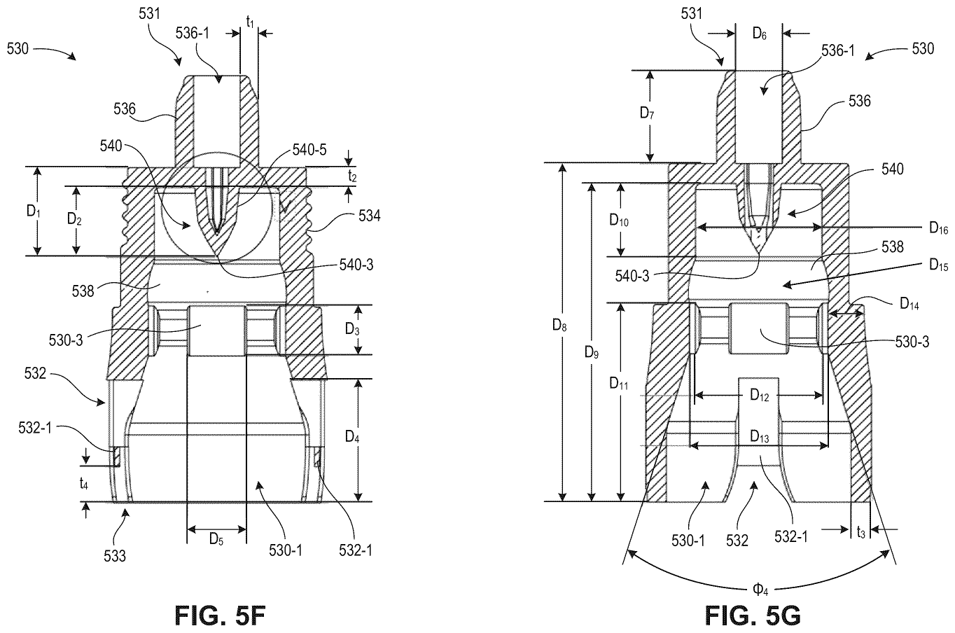

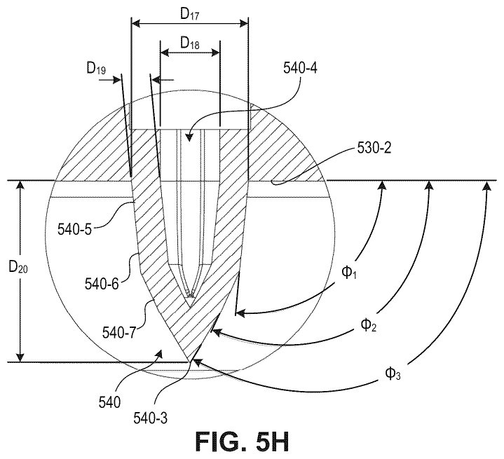

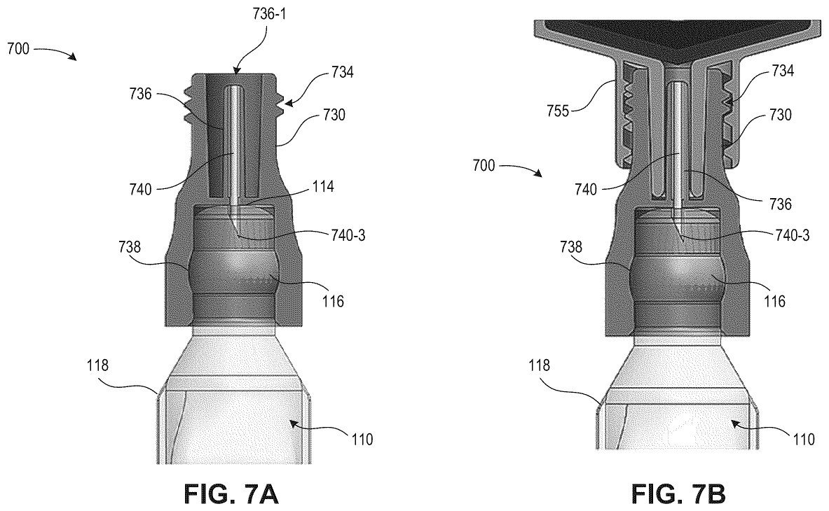

The threads 334 may comprise any suitable coupling features that are operable to selectively couple the distal end of the piercing connector 330 to an additional device or object (not shown; e.g., the housing 250 of FIGS. 2A-2C). For example, in some embodiments, one or more luer-style thread protrusions (e.g., a male-type luer lock connector or a female-type luer lock connector) can comprise and/or define the threads 334. In the illustrated example, threads 334 are disposed on a radially-inner surface of an axially-extending annular member at least partially surrounding the outlet conduit 336. Alternatively or additionally, in some embodiments, the threads can be disposed on a radially-outer surface of the outlet conduit 336, and/or the axially-extending annular member can be omitted (e.g., similar to the configurations shown in FIGS. 5A-5H or 7A-7B).

According to some embodiments, the piercing connector 330 may comprise a mounting recess 338 (e.g., a scalloped surface) formed within the inlet port 330-1 and configured to accept the mounting portion 116. The piercing connector 330 may be axially engaged to couple with the BFS vial 110 via application of a mating axial force, for example, such as via an urging of the piercing connector 330 (and/or the inlet port 330-1 thereof) onto the neck 112 of the BFS vial 110. In some embodiments, the piercing connector 330 (and/or the inlet port 330-1 thereof) may accept and/or selectively couple to the mounting portion 116, thereby removably coupling the BFS vial 110 and the piercing connector 330. In some embodiments, uncoupling of the BFS vial 110 and the piercing connector 330 may be mechanically prohibited.

In some embodiments, the piercing element 340 may comprise an “X” and/or cross-shaped configuration (e.g., with one cross-member protruding higher than the other) and/or may be integral to the piercing connector 330. In some embodiments, the piercing element 340 may be constructed of a different material than the piercing connector 330 and/or may be coupled to the piercing connector 330. In some embodiments, the piercing element 340 comprises and/or defines one or more fluid conduits that can connect and/or provide fluid communication between outlet port 336-1 and inlet port 330-1 (or portions thereof). In some embodiments, mounting recess 338 may be axially distanced from a lowermost tip of the piercing element 340 by a distance designed to cause the piercing element 340 to extend through seal 114 (at least partially) of the BFS vial 110 in the case that the coupling portion 116 of the neck 112 of the BFS vial 110 is seated within mounting recess 338.

According to some embodiments, the BFS vial 110 may be engaged with the piercing connector 330 in two positions or stages. In a first stage or position as depicted in FIG. 3A, for example, the BFS vial 110 may be partially inserted into the piercing connector 330 such that the fluid seal 114 is positioned adjacent to and/or aligned with a piercing element 340 of the piercing connector 330. In some embodiments, the first stage of FIG. 3A can be a state of assembly as delivered from a manufacturing facility for use in the field with minimal assembly (e.g., with piercing element 340 adjacent to but not breaching fluid seal 114), or a state of assembly of individual components by a user in preparation for administration. In a second stage or position as depicted in FIG. 3B, the BFS vial 110 may be fully inserted into the piercing connector 330, such that the piercing element 340 pierces, breaks, or otherwise extends through the fluid seal 114 and/or such that the mounting flange 116 is retained by the mounting recess 338. In some embodiments, the breach of fluid seal 114 by the piercing element 340 can allow fluidic access to the contents of the BFS vial 110 (e.g., the fluid retained within reservoir 120 and/or reservoir 122) and/or can establish fluid communication between the interior of the BFS vial 110 and the outlet port 336-1.

In some embodiments, fewer or more components 110-166, 230-280, 300-340 and/or various configurations of the depicted components 110-166, 230-280, 300-340 may be included in pre-filled medical delivery assembly 300 without deviating from the scope of embodiments described herein. In some embodiments, the components 110-166, 230-280, 300-340 may be similar in configuration and/or functionality to similarly named and/or numbered components as described herein.

### V. Second Exemplary Piercing Connectors

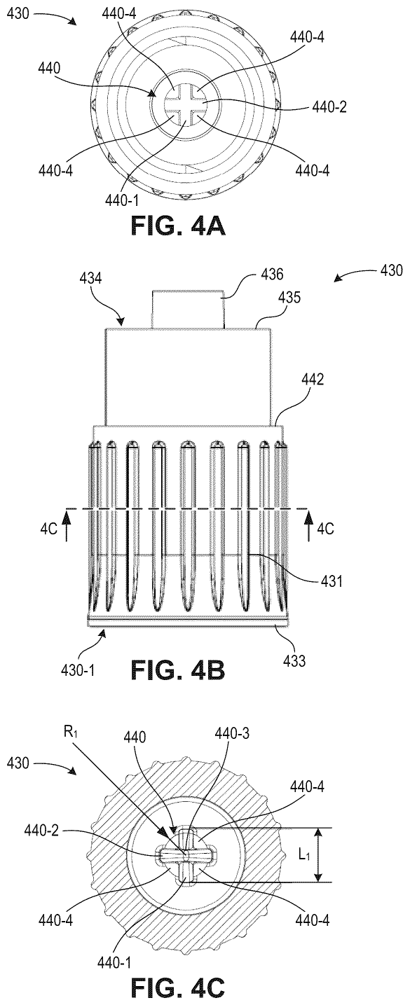

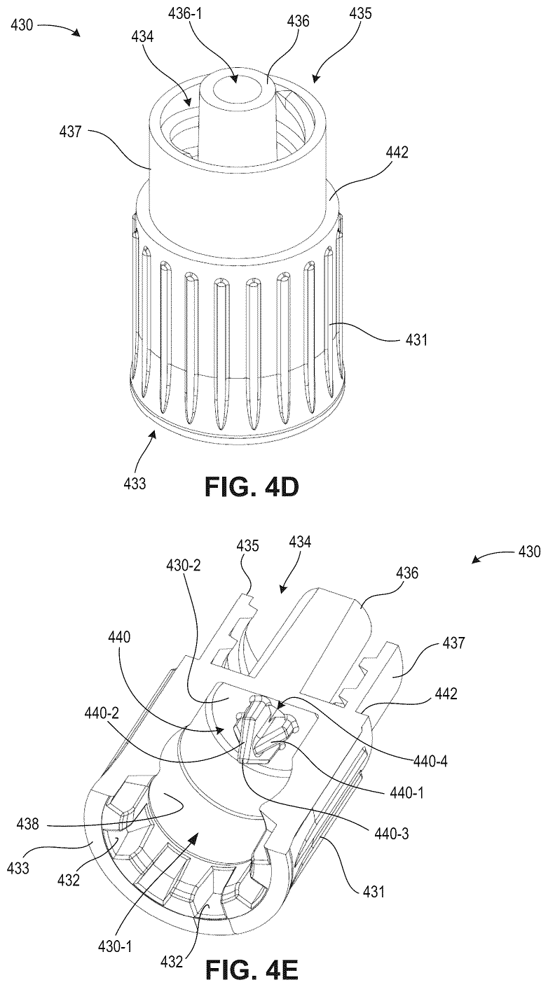

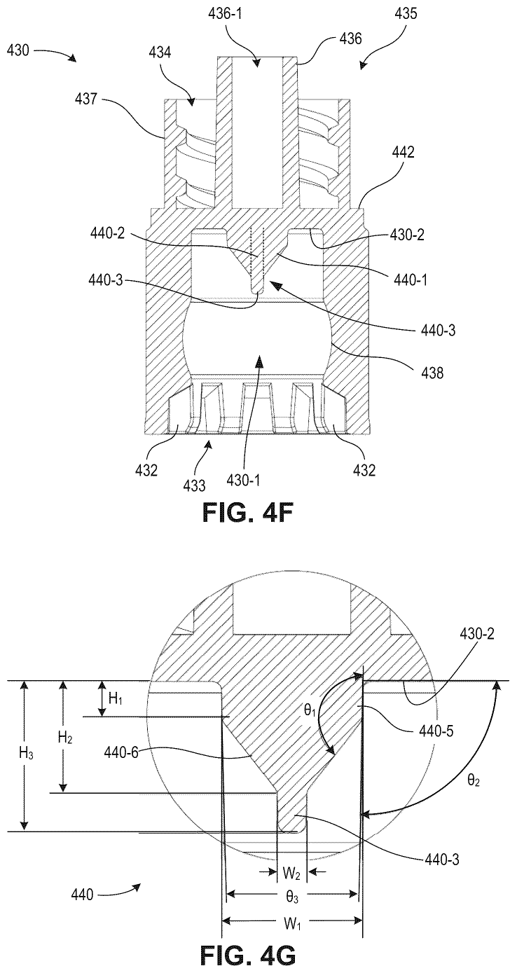

FIGS. 4A-4E show various views of a piercing connector 430 for accessing pre-filled vials (e.g., BFS vial 110 and/or BFS vial 150) according to one or more embodiments. The piercing connector 430 may comprise similar features and/or configurations and/or may be similar and/or be used similar to any of connectors 130, 330, 530, 630, and 730 of FIGS. 2A-2C, 3A-3B, 5A-5H, 6A-6F, and 7A-7B, respectively. The piercing connector 430 may comprise, for example, a generally cylindrical body defining an inlet or interior volume 430-1 open at a first or proximal end and terminating at a chamber end wall 430-2. In some embodiments, the piercing connector 430 may comprise one or more anti-rotation coupling features 432 disposed at the first end and/or threads 434 disposed at a second or distal end thereof. According to some embodiments, the piercing connector 430 may comprise or define a nozzle 436 (also referred to herein as neck, stem, lumen, or conduit) defining an interior bore or outlet channel 436-1. According to some embodiments, the piercing connector 430 may comprise an internal groove or seat 438 that is cooperatively sized and configured to receive a mounting flange of a BFS vial (neither shown; e.g., the mounting portion 116, 156 of the BFS vials 110, 150 of FIGS. 1A-1D).

In some embodiments, the piercing connector 430 may comprise and/or define a piercing element 440. The piercing connector 430 may comprise an angled and/or sharpened protrusion of “hard” plastic, for example, that is disposed within the interior volume 430-1 such that in the case that a BFS vial is seated in the interior volume 430-1 and/or a mounting flange thereof is seated in the seat 438, the piercing element 440 may engage with and puncture a seal of the BFS vial (not shown; e.g., the fluid seal 114, 154 of the BFS vials 110, 150 of FIGS. 1A-1D). According to some embodiments, the piercing connector 430 may comprise a first or lower shaped portion 440-1 (e.g., a first crossmember) that joins with a second or upper shaped portion 440-2 (e.g., a second crossmember) to form the structure of the piercing connector 430 and/or to define a point 440-3 (also referred to herein as a tip). As depicted in FIGS. 4A-4C, for example, the lower shaped portion 440-1 may comprise an axially angled protrusion that intersects with the upper shaped portion 440-2 to form a cross-shape that terminates axially in the interior volume 430-1 (e.g., oriented toward the proximal end 433) in the point 440-3. In some embodiments, the upper shaped portion 440-2 may be considered to be a main member that is buttressed on its opposite lateral sides by perpendicularly-extending mirrored halves of the lower shaped portion 440-1. In some embodiments, the cross-shape of the piercing element 440 may define channels 440-4 (e.g., four (4), as depicted, although fewer or greater number of channels 440-4 are also possible). In some embodiments, one, some, or all of channels 440-4 can communicate and/or connect between the interior volume 430-1 and the outlet channel 436-1. In such a manner, for example, in the case that the point 440-3 is engaged to pierce a fluid seal of a BFS vial, fluid from the BFS vial may flow through the channels 440-4 and into the outlet channel 436-1.

In some embodiments, the piercing element 440 may comprise and/or define an axial projection 440-5 that emanates from the chamber end wall 430-2 at an angle θ₂. According to some embodiments, the piercing element 440 may comprise and/or define an angled portion 440-6 that emanates radially inward and axially away from the axial projection 440-5 at an angle θ₁. In some embodiments, the point 440-3 may be disposed and/or formed at a junction of two symmetrical and opposed angled portions 440-6 and/or may extend axially therefrom. According to some embodiments, the point 440-3 may have and/or define a width, W₂, and/or diameter, R₁. In some embodiments, the piercing element 440 may comprise a third axial length, H₃, the axial projection 440-5 and the angled portion 440-6 may together comprise a second axial length, H₂, and/or the axial projection 440-5 may comprise a first axial length, H₁. According to some embodiments, the piercing element 440 may comprise a width, W₁. Table 1 below provides exemplary dimensions for labeled features of the piercing connector 430, in particular, aspects related to the piercing element 440; however, dimensions other than those listed in Table 1 are also possible according to one or more contemplated embodiments.

TABLE 1
Exemplary values for dimensions of
piercing connector of FIGS. 4A-4G.

| FIG. | Exemplary | |
|---|---|---|
| Label | Value | |
| R₁ | 1.3 | mm |
| L₁ | 3 | mm |
| H₁ | 0.8 | mm |
| H₂ | 2.4 | mm |
| H₃ | 3.2 | mm |
| W₁ | 3 | mm |
| W₂ | 0.6 | mm |

| | |
|---|---|
| Θ₁ | 143° |
| Θ₂ | 91° |
| Θ₃ | 2° |

In some embodiments, the outlet channel 436-1 may extend through and/or be at least partially defined by the piercing element 440 and/or the channels 440-4 thereof such that fluid released from the BFS vial due to a piercing of the seal by the piercing element 440 may be administered through (e.g., selectively) the outlet channel 436-1. According to some embodiments, the body of connector 430 may be sized to permit a user to have a suitable gripping surface (e.g., a “hard” plastic gripping surface) such that connector 430 may be utilized to attached/engage various components such as an administration member (not shown) with the BFS vial and/or “soft” plastic neck thereof (e.g., without requiring such gripping forces to be applied directly to the BFS vial, which would cause deformation thereof). The length of the body of the connector 430 may, for example, be sized between twelve millimeters (12 mm) and twenty millimeters (20 mm).

According to some embodiments, the one or more anti-rotation coupling features 432 may comprise one or more axial slits. Alternatively or additionally, in some embodiments, the one or more anti-rotation coupling features 432 may comprise a plurality of indents, seats, or grooves, such as depicted as a “castle” nut configuration. In the case of the slit configuration of the one or more anti-rotation coupling features 432, such features may engage with and/or house or retain (e.g., prevent rotation of) a bottle flange of the BFS vial (not shown; e.g., the body flanges 118, 158 of BFS vials 110, 150 of FIGS. 1A-1D). In the case of the castle nut configuration of the one or more anti-rotation coupling features 432, such features may engage with and/or house or retain (e.g., prevent rotation of) a portion of a bottle flange of the BFS vial (not shown; e.g., the body flanges 118, 158 of BFS vials 110, 150 of FIGS. 1A-1D).

According to some embodiments, the connector 430 may comprise and/or define an annular coupling member 437 and/or an external flange. In some embodiments, the annular coupling member 437 can comprise and/or define threads, for example, for coupling an additional device or object (not shown; e.g., a component of an administration assembly, such as housing 250 of FIGS. 2A-2C). Alternatively or additionally, external flange may, for example, permit one or more objects (not shown) to be coupled to the distal or outlet (e.g., upper, as shown) end 435 of the connector 430 but to be restrained from traveling onto the connector 430 beyond protrusion 442 (also referred to as a step, lip, or ledge) of the external flange. In some embodiments, the protrusion 442 of the external flange may act as a seat for a portion of a cap and/or housing (not shown; e.g., housing 250 and/or cap 280 of FIGS. 2A-2C).

In some embodiments, fewer or more components 430-442 and/or various configurations of the depicted components 430-442 may be included in the connector 430 without deviating from the scope of embodiments described herein. In some embodiments, the components 430-442 may be similar in configuration and/or functionality to similarly named and/or numbered components as described herein.

### VI. Third Exemplary Piercing Connectors

FIGS. 5A-5H show various views of a piercing connector 530 for accessing pre-filled vials (not shown) according to some embodiments. The piercing connector 530 may comprise similar features and/or configurations and/or may be similar and/or be used similar to any of connectors 130, 330, 430, 630, and 730 of FIGS. 2A-2C, 3A-3B, 4A-4G, 6A-6F, and 7A-7B, respectively. The piercing connector 530 may comprise, for example, a generally cylindrical body 537 defining an inlet or interior volume 530-1 open at a first or proximal end 533 and terminating at a chamber end wall 530-2. In some embodiments, an external surface of the body 537 of the piercing connector 530 can be knurled (e.g., comprising one or more protrusions), for example, to provide a gripping surface for a user. In some embodiments, a pathway into the interior volume 530-1 may be modified from a simply circular cross-section to provide for easier entry of an inserted mounting flange of a BFS vial (not shown). As depicted in FIG. 5A, for example, the interior passage may comprise a one or more radially spaced undercuts 530-3 that locally increase the interior diameter of the passage to provide less friction to a mounting flange urged axially into the interior volume 530-1. According to some embodiments, the piercing connector 530 may comprise an internal groove or seat 538 that is cooperatively sized and configured to receive a mounting flange of a BFS vial (neither shown; e.g., the mounting portion 116, 156 of the BFS vials 110, 150 of FIGS. 1A-1D).

In some embodiments, the piercing connector 530 may comprise and/or define a piercing element 540. The piercing element 540 may comprise an angled and/or sharpened protrusion of “hard” plastic, for example, that is disposed within the interior volume 530-1 such that in the case that a BFS vial is seated in the interior volume 530-1 and/or a mounting flange thereof is seated in the seat 538, the piercing element 540 may engage with and puncture a seal of the BFS vial (not shown; e.g., the fluid seal 114, 154 of the BFS vials 110, 150 of FIGS. 1A-1D). In some embodiments, the piercing connector 530 may comprise one or more anti-rotation coupling features 532 (and respective frangible positioning elements 532-1) disposed at the first end 533. According to some embodiments, the piercing connector 530 may comprise or define a nozzle 536 (also referred to herein as a neck, stem, lumen, or conduit) defining an interior bore or outlet channel 536-1. In some embodiments, an external surface of the body of the piercing connector 530 can comprise one or more threads 534, for example, on an external surface of the connector body between a tip 540-3 of a piercing element 540 and a second or distal end 531 along an axial direction of the piercing connector 530.

According to some embodiments, the piercing element 540 may comprise a tapered conical protrusion comprising a piercing tip 540-3 and one or more fluid bores 540-4. In some embodiments, and as depicted in FIGS. 5F-5H, the piercing element 540 may comprise and/or define a first (e.g., conical or frustoconical) projection 540-5 that emanates axially away from the chamber end wall 530-2 at a first angle, ϕ₁. According to some embodiments, the piercing element 540 may comprise and/or define a second (e.g., conical or frustoconical) projection 540-6 that emanates axially away from the first projection 540-5 to form a second angle, ϕ₂, with the chamber end wall 530-2. In some embodiments, the piercing element 540 may comprise and/or define a third (e.g., conical or frustoconical) projection 540-7 that emanates axially away from the second projection 540-6 to form a third angle, ϕ₃, with the chamber end wall 530-2. According to some embodiments, the point 540-3 may be disposed and/or formed at an axial terminus of the third projection 540-7. In some embodiments, the piercing element 540 may comprise a first axial length D₂₀ and/or a wall thickness D₁₉, the tip 540-3 may have a width and/or diameter Du, and/or the fluid bores 540-4 may comprise a bore diameter, D₁₈. Table 2 below provides exemplary dimensions for labeled features of the piercing connector 530, in particular, aspects related to the piercing element 540; however, dimensions other than those listed in Table 2 are also possible according to one or more contemplated embodiments.

In some embodiments, the outlet channel 536-1 may extend through and/or be at least partially defined by the piercing element 540 and/or the fluid bores 540-4 thereof such that fluid released from the BFS vial due to a piercing of the seal by piercing element 540 may be administered through (e.g., selectively) outlet channel 536-1. According to some embodiments, the body of the connector 530 may be sized to permit a user to have a suitable gripping surface (e.g., a “hard” plastic gripping surface) such that connector 530 may be utilized to attached/engage various components such as an administration member (not shown) with the BFS vial and/or “soft” plastic neck thereof (e.g., without requiring such gripping forces to be applied directly to the BFS vial, which would cause deformation thereof). The length of the body of connector 530 may, for example, be sized between twelve millimeters (12 mm) and twenty millimeters (20 mm).

TABLE 2
Exemplary values for dimensions of
piercing connector of FIGS. 5A-5H.

| FIG. | Exemplary | |
|---|---|---|
| Label | Value | |
| t₁ | 0.93 | mm |
| t2 | 1 | mm |
| t3 | 0.96 | mm |
| t4 | 1.82 | |
| D1 | 4.50 | mm |
| D2 | 3.50 | mm |
| D3 | 2.51 | mm |
| D4 | 6.17 | mm |
| D5 | 3 | mm |
| D6 | 2.33 | mm |
| D7 | 4.61 | mm |
| D8 | 16.85 | mm |
| D9 | 15.85 | mm |
| D10 | 3.65 | mm |
| D11 | 9.89 | mm |
| D12 | 6.32 | mm |
| D13 | 6.90 | mm |
| D14 | 1.80 | mm |
| D15 | 4.14 | mm |
| D16 | 6.30 | mm |
| D17 | 2.25 | mm |
| D18 | 1.14 | mm |
| D19 | 0.55 | mm |
| D20 | 3.50 | mm |

| | |
|---|---|
| Φ1 | 95.71° |
| Φ2 | 115.02° |
| Φ3 | 120.96° |
| Φ4 | 34.62° |

According to some embodiments, the one or more anti-rotation coupling features 532 may comprise one or more axial slits. In some embodiments, the anti-rotation coupling features 532 may engage with and/or house or retain (e.g., prevent rotation of) a bottle flange of the BFS vial (not shown; e.g., the body flanges 118, 158 of BFS vials 110, 150 of FIGS. 1A-1D). In some embodiments, the BFS vial may be engaged with the connector 530 in two positions or stages. In a first stage or position, for example, the BFS vial may be partially inserted into the connector 530 such that the fluid seal is positioned adjacent to and/or aligned with the piercing element 540. According to some embodiments, attainment and/or maintenance of the first position may be facilitated by the frangible positioning elements 532-1. The BFS vial may be inserted into the connector 530 to an extent, for example, until the leading edges of the bottle flanges enter into the anti-rotation features 532 (e.g., axial slits, as depicted), make contact with, and/or are blocked by the frangible positioning elements 532-1. The bottle flanges may, in the first position be disposed a first distance into anti-rotation features 532 and/or be disposed a first distance from the terminus of anti-rotation features 532. In some embodiments, contact between frangible positioning elements 532-1 and bottle flanges may define the first position between the BFS vial and the connector 530 (and/or the first stage thereof).

According to some embodiments, in a second stage or position the BFS vial may be fully inserted into the connector 530 such that the piercing element 540 pierces the fluid seal and/or such that the mounting flange is retained by and/or seated in the seat 538. In some embodiments, an axial force may be applied to urge the BFS vial into the connector 530 such that the leading edges of the bottle flanges sever, fracture, and/or otherwise cause the frangible positioning elements 532-1 to fail. In such embodiments, the bottle flanges may then travel deeper into the anti-rotation features 532 than originally positioned in the first position. The bottle flanges may, in the second position, be disposed a first distance into the anti-rotation features 532 and/or be disposed a second distance from the terminus of the anti-rotation features 532. According to some embodiments, a step for employing a pre-filled medical delivery assembly may be to apply axial compressive force urging the BFS vial axially into the connector 530, severing the frangible positioning elements 532-1, and engaging the mounting flange with the seat 538.

According to some embodiments, the connector 530 may comprise or define an external flange 542. The external flange 542 may, for example, permit one or more objects (not shown) to be coupled to the distal or outlet (e.g., upper, as shown) end of the connector 530 but to be restrained from traveling onto the connector 530 beyond the protrusion of the external flange 542. In some embodiments, the external flange 542 may act as a seat for a portion of a cap and/or housing (not shown; e.g., housing 250 and/or cap 280 of FIGS. 2A-2C).

In some embodiments, fewer or more components 530-542 and/or various configurations of the depicted components 530-542 may be included in the connector 530 without deviating from the scope of embodiments described herein. In some embodiments, the components 530-542 may be similar in configuration and/or functionality to similarly named and/or numbered components as described herein.

### VII. Fourth Exemplary Piercing Connectors and Assemblies Including Such Connectors

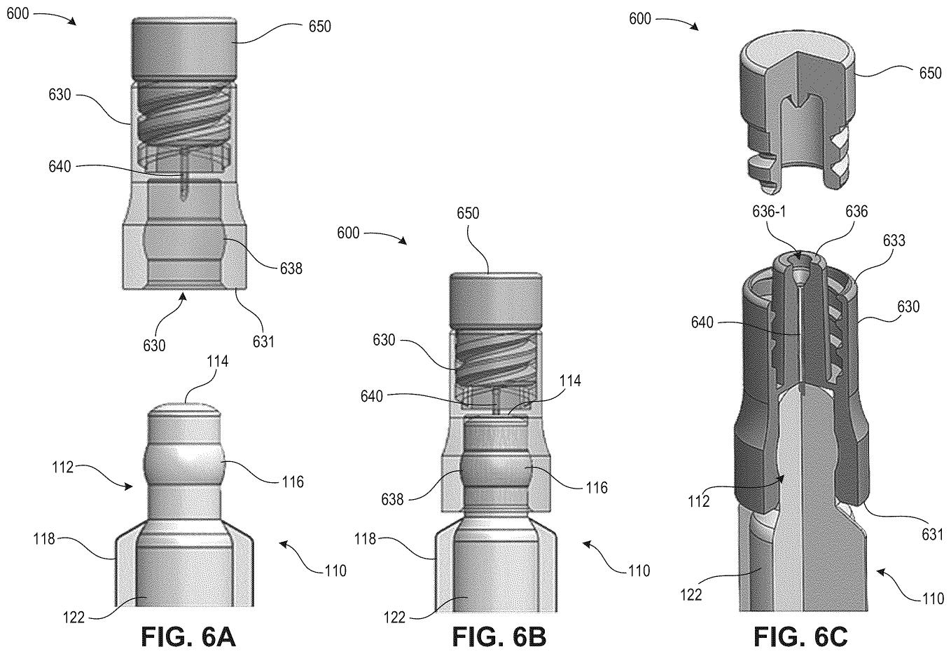

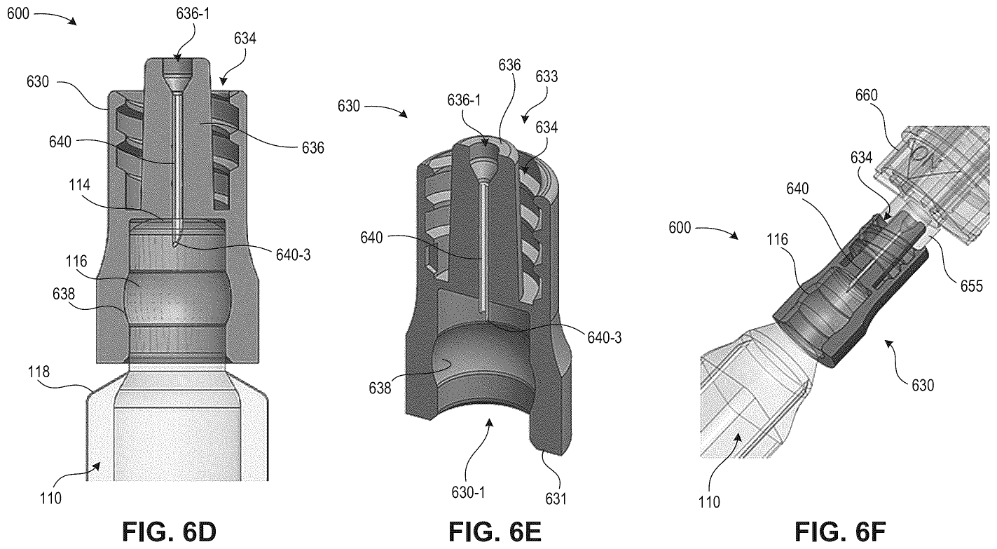

FIGS. 6A-6F show various views of a system 600 for piercing pre-filled vials according to some embodiments. In some embodiments, the system 600 may comprise a piercing connector 630, outlet cap 650, and/or BFS vial 110 (e.g., only a portion of which is depicted) or any other sealed fluid-filled plastic container. In some embodiments, the neck 112 of the BFS vial 110 may be engaged with and/or coupled to piercing connector 630 defining an interior chamber 630-1. In some embodiments, the piercing connector 630 may comprise an internal mounting feature such as a seat or groove 638 (within chamber 630-1), which can be shaped to accept and/or retain the mounting portion 116 upon insertion of the neck 112 of the BFS vial 110 into the piercing connector 630, for example, into chamber 630-1 via inlet end 631. In some embodiments, the piercing connector 630 may comprise a piercing element 640 disposed to pierce the seal 114 in the case that the BFS vial 110 is inserted into the piercing connector 630. For example, the piercing element 640 may comprise a tube and/or projection beveled to form a point 640-3 configured and arranged to pierce seal 114, as shown in FIGS. 6B-6D. According to some embodiments, the piercing element 640 may comprise a metal tube that is inserted and/or formed into the outlet projection 636 and extends into the chamber 630-1 to engage with the seal 114.

In some embodiments, outlet projection 636 can comprise and/or define an outlet channel 636-1. In some embodiments, the piercing connector 630 may comprise and/or define external threads 634 disposed on a radially-inner surface of annular flange surrounding outlet projection 636 (also referred to herein as a nozzle, neck, stem, lumen, or conduit). In some embodiments, one or more luer-style thread protrusions (e.g., a male-type luer lock connector or a female-type luer lock connector) can comprise and/or define the threads 634. In its initial configuration of system 600, an outlet cap 650 can be coupled to outlet end 633 via threads 634 of the annular flange. In some embodiments, the piercing connector 630 can be used to selectively prepare the BFS vial 110 for engagement with one or more other devices 660, such as a syringe (e.g., via housing 250 of FIGS. 2A-2C) and/or an intravenous (IV) therapy device (e.g., an infusion apparatus, IV bag, etc.). For example, the cap 650 can be removed from outlet end 633 of the connector 630, as shown in FIG. 6C (e.g., by unscrewing the cap 650 from threads 634), and the other device 660 can be attached to the outlet projection 636, as shown in FIG. 6F (e.g., by screwing the luer lock coupler 655 onto threads 634), for example, to provide fluid communication with an interior of the BFS vial 110 and the other device 660 via connector 630.

In some embodiments, fewer or more components 110-166, 630-660 and/or various configurations of the depicted components 110-166, 630-660 may be included in the system 600 without deviating from the scope of embodiments described herein. In some embodiments, the components 110-166, 630-660 may be similar in configuration and/or functionality to similarly named and/or numbered components as described herein.

### VIII. Fifth Exemplary Piercing Connectors and Assemblies Including Such Connectors

FIGS. 7A-7B show various views of a system 700 for piercing pre-filled vials according to some embodiments. In some embodiments, the system 700 may comprise a piercing connector 730 and/or BFS vial 110 (e.g., only a portion of which is depicted) or any other sealed fluid-filled plastic container. In some embodiments, the neck 112 of the BFS vial 110 may be engaged with and/or coupled to piercing connector 730 defining an interior chamber (not separately labeled). In some embodiments, the piercing connector 730 may comprise an internal mounting feature such as a seat or groove 738, which can be shaped to accept and/or retain the mounting portion 116 upon insertion of the neck 112 of the BFS vial 110 into the piercing connector 730. In some embodiments, the piercing connector 730 may comprise a piercing element 740 disposed to pierce the seal 114 in the case that the BFS vial 110 is inserted into the piercing connector 730. For example, the piercing element 740 may comprise a tube and/or projection beveled to form a point 740-3 configured and arranged to pierce seal 114, as shown in FIGS. 7A-7B. According to some embodiments, the piercing element 740 may comprise a metal tube that is inserted and/or formed into the outlet projection 736 and extends into the chamber 730-1 to engage with the seal 114.

In some embodiments, outlet projection 736 can comprise and/or define an outlet channel 736-1. In some embodiments, the configuration and operation of the piercing connector 730 can be substantially similar to that of piercing connector 630 of FIGS. 6A-6F, except for the annular flange and the location of threads, which may permit different types and/or configurations of couplings, as is or becomes desirable and/or practicable. For example, the piercing connector 730 may comprise and/or define external threads 734 disposed on a radially-outer surface of outlet projection 736 (also referred to herein as a nozzle, neck, stem, lumen, or conduit). In some embodiments, one or more luer-style thread protrusions (e.g., a male-type luer lock connector or a female-type luer lock connector) can comprise and/or define the threads 734. In some embodiments, the piercing connector 730 can be used to selectively prepare the BFS vial 110 for engagement with one or more other devices 755, such as a syringe (e.g., via housing 250 of FIGS. 2A-2C) and/or an intravenous (IV) therapy device (e.g., an infusion apparatus, IV bag, etc.). For example, the other device 755 can be attached to the outlet projection 736, as shown in FIG. 7B (e.g., by screwing the device 755 into threads 734), for example, to provide fluid communication with an interior of the BFS vial 110 and the other device 755 via connector 730.

In some embodiments, fewer or more components 110-166, 730-755 and/or various configurations of the depicted components 110-166, 730-755 may be included in the system 700 without deviating from the scope of embodiments described herein. In some embodiments, the components 110-166, 730-755 may be similar in configuration and/or functionality to similarly named and/or numbered components as described herein.

### IX. Additional Examples of the Disclosed Technology

In view of the above described implementations of the disclosed subject matter, this application discloses the additional examples in the clauses enumerated below. It should be noted that one feature of a clause in isolation, or more than one feature of the clause taken in combination, and, optionally, in combination with one or more features of one or more further clauses are further examples also falling within the disclosure of this application.

Clause 1. A pre-filled medical delivery assembly comprising:
(i) a blow-fill-seal (BFS) vial having first and second longitudinal ends, the BFS vial comprising:
   at least one reservoir having a fluid therein; and
   a longitudinally-extending neck portion having a fluid seal at the first longitudinal end of the BFS vial; and (ii) a connector comprising:
   an inlet port connecting to a first interior volume;
   an outlet port connecting to a second interior volume in fluid communication with the first interior volume; and
   a piercing element having a least a piercing tip disposed within the first interior volume,
wherein the neck portion of the BFS vial extends through the inlet port into the first interior volume such that at least part of the piercing element breaches and extends through the fluid seal.

Clause 2. A pre-filled medical delivery assembly comprising:
(i) a blow-fill-seal (BFS) vial having first and second longitudinal ends, the BFS vial comprising:
   at least one reservoir having a fluid therein; and
   a longitudinally-extending neck portion having a fluid seal at the first longitudinal end of the BFS vial; and (ii) a connector comprising:
   an inlet port connecting to a first interior volume;
   an outlet port connecting to a second interior volume in fluid communication with the first interior volume; and
   means for piercing the fluid seal of the BFS vial,
wherein the neck portion of the BFS vial extends through the inlet port into the first interior volume.

Clause 3. The pre-filled medical delivery assembly of any clause or example herein, in particular, any one of clauses 1-2, wherein the inlet port and the outlet port are substantially coaxial and disposed at opposite longitudinal ends of the connector.

Clause 4. The pre-filled medical delivery assembly of any clause or example herein, in particular, any one of clauses 1-3, wherein the connector comprises a longitudinally-extending nozzle that forms the outlet port.

Clause 5. The pre-filled medical delivery assembly of any clause or example herein, in particular, clause 4, wherein the connector further comprises a longitudinally-extending annular flange that at least partially surrounds the nozzle.

Clause 6. The pre-filled medical delivery assembly of any clause or example herein, in particular, clause 5, wherein a radially-inner surface of the annular flange, a radially-outer surface of the annular flange, or both comprise threads.

Clause 7. The pre-filled medical delivery assembly of any clause or example herein, in particular, any one of clauses 5-6, wherein the annular flange, the nozzle, or both form a luer lock connection member.

Clause 8. The pre-filled medical delivery assembly of any clause or example herein, in particular, any one of clauses 4-7, wherein a radially-outer surface of the nozzle comprises threads.

Clause 9. The pre-filled medical delivery assembly of any clause or example herein, in particular, any one of clauses 4-8, wherein the nozzle forms a luer lock connection member.

Clause 10. The pre-filled medical delivery assembly of any clause or example herein, in particular, any one of clauses 1-9, wherein the piercing element or the means for piercing defines at least a portion of a flow path from an interior of the neck portion to the second interior volume.

Clause 11. The pre-filled medical delivery assembly of any clause or example herein, in particular, any one of clauses 1-10, wherein an interior sidewall of the connector that forms the first interior volume comprises a mating feature, and/or the neck portion of the BFS vial comprises a laterally-protruding portion that is received in the mating feature so as to couple the BFS vial to the connector.

Clause 12. The pre-filled medical delivery assembly of any clause or example herein, in particular, clause 11, wherein the mating feature comprises a scalloped portion of the interior sidewall, and/or each laterally-protruding portion has a toroidal shape in a side view.

Clause 13. The pre-filled medical delivery assembly of any clause or example herein, in particular, any one of clauses 1-10, wherein the connector and the neck portion of the BFS vial comprise means for coupling together.

Clause 14. The pre-filled medical delivery assembly of any clause or example herein, in particular, any one of clauses 1-13, wherein:
the BFS vial further comprises flange portions disposed on opposite lateral sides of a body portion of the BFS vial and proximal to a junction of the body portion with the neck portion; and
the connector comprises a body portion that defines the inlet port, the body portion having one or more anti-rotation coupling features that interact with the flange portions of the BFS vial to inhibit rotation of the BFS vial with respect to the connector.

Clause 15. The pre-filled medical delivery assembly of any clause or example herein, in particular, clause 14, wherein the anti-rotation coupling features comprises one or more longitudinally-extending slots through a sidewall of the body portion, one or more laterally-indented portions disposed around a circumference of the inlet port, or any combination of the foregoing.

Clause 16. The pre-filled medical delivery assembly of any clause or example herein, in particular, any one of clauses 1-13, wherein the connector comprises means for inhibiting rotation of the BFS vial with respect to the connector.

Clause 17. The pre-filled medical delivery assembly of any clause or example herein, in particular, any one of clauses 1-16, wherein the piercing element or the means for piercing comprises at least first and second crossmembers.

Clause 18. The pre-filled medical delivery assembly of any clause or example herein, in particular, clause 17, wherein the at least first and second crossmembers are arranged to form a cross shape.

Clause 19. The pre-filled medical delivery assembly of any clause or example herein, in particular, any one of clauses 17-18, wherein the second crossmembers are disposed on opposite sides of and extend orthogonal to the first crossmember, and/or the first crossmember extends further along a longitudinal direction than the second crossmembers such that a central portion of the first crossmember forms the piercing tip.

Clause 20. The pre-filled medical delivery assembly of any clause or example herein, in particular, any one of clauses 17-19, wherein the first interior volume is defined at least in part by a chamber end wall of the connector, and/or the piercing element longitudinally extends from the chamber end wall toward the inlet port, and/or the chamber end wall comprises one or more openings fluidically connecting the first interior volume to the second interior volume.

Clause 21. The pre-filled medical delivery assembly of any clause or example herein, in particular, clause 20, wherein the chamber end wall has a plurality of the openings, each opening corresponding to and connecting with a respective quadrant defined by the cross shape of the piercing element.

Clause 22. The pre-filled medical delivery assembly of any clause or example herein, in particular, any one of clauses 1-21, wherein the piercing element or the means for piercing is integrally formed with the connector, and/or the connector and the piercing element or the means for piercing are formed of a plastic having a hardness greater than that of the BFS vial.

Clause 23. The pre-filled medical delivery assembly of any clause or example herein, in particular, any one of clauses 17-22, wherein the first and second crossmembers of the piercing element are substantially solid, such that the fluid from the at least one reservoir flows around the crossmembers en route to the second interior volume.

Clause 24. The pre-filled medical delivery assembly of any clause or example herein, in particular, any one of clauses 1-16, wherein the piercing element or the means for piercing comprises a conical or frustoconical member.

Clause 25. The pre-filled medical delivery assembly of any clause or example herein, in particular, clause 24, wherein the conical or frustoconical member comprises one or more lumens longitudinally extending therethrough so as to fluidically connect the first interior volume to the second interior volume.

Clause 26. The pre-filled medical delivery assembly of any clause or example herein, in particular, any one of clauses 24-25, wherein the first interior volume is defined at least in part by a chamber end wall of the connector, and/or the conical or frustoconical member longitudinally extends from the chamber end wall toward the inlet port.

Clause 27. The pre-filled medical delivery assembly of any clause or example herein, in particular, any one of clauses 24-26, wherein the conical or frustoconical member is integrally formed with the connector, and/or the connector and the conical or frustoconical member are formed of a plastic having a hardness greater than that of the BFS vial.

Clause 28. The pre-filled medical delivery assembly of any clause or example herein, in particular, any one of clauses 1-16, wherein the piercing element or the means for piercing comprises a hollow metal tube, and/or the piercing tip is formed by a beveled edge of the metal tube.

Clause 29. The pre-filled medical delivery assembly of any clause or example herein, in particular, any clause 28, wherein the connector comprises a longitudinally-extending nozzle that forms the outlet port, and the hollow metal tube is at least partially embedded within the nozzle.

Clause 30. The pre-filled medical delivery assembly of any clause or example herein, in particular, any one of clauses 1-29, wherein the connector is formed of a plastic having a hardness greater than that of the BFS vial.

Clause 31. The pre-filled medical delivery assembly of any clause or example herein, in particular, any one of clauses 1-30, further comprising:
(iii) an administration assembly comprising:
   a hub coupled to the outlet port of the connector; and
   at least one administration conduit having an outlet at an end thereof longitudinally spaced from the hub and being in fluid communication with the second interior volume.

Clause 32. The pre-filled medical delivery assembly of any clause or example herein, in particular, clause 31, wherein the hub is coupled to the outlet port via a luer lock connection.

Clause 33. The pre-filled medical delivery assembly of any clause or example herein, in particular, any one of clauses 31-32, wherein at least part of the administration assembly is formed separate from and constructed to be coupled to the connector, or at least part of the administration assembly is integrally formed with the connector

Clause 34. The pre-filled medical delivery assembly of any clause or example herein, in particular, any one of clauses 31-33, wherein the at least one administration conduit comprises a needle or cannula.

Clause 35. The pre-filled medical delivery assembly of any clause or example herein, in particular, clause 34, wherein the needle has a length in a range of 0.5 mm to 4 mm, inclusive, or in a range of 4 mm to 15 mm, inclusive, or in a range of 15 mm to 30 mm, inclusive.

Clause 36. The pre-filled medical delivery assembly of any clause or example herein, in particular, any one of clauses 31-33, wherein the outlet of the at least one administration conduit is formed as a nozzle configured to facilitate dispersion of the fluid from the BFS vial into a spray, or the outlet of the at least one administration conduit is formed as a nozzle configured to facilitate dispersion of the fluid from the BFS vial into one or more droplets.

Clause 37. The pre-filled medical delivery assembly of any clause or example herein, in particular, any one of clauses 1-30, further comprising (iii) an intravenous (IV) therapy device coupled to the outlet port of the connector.

Clause 38. The pre-filled medical delivery assembly of any clause or example herein, in particular, clause 37, wherein the IV therapy device is coupled to the outlet port via a luer lock connection.

Clause 39. The pre-filled medical delivery assembly of any clause or example herein, in particular, any one of clauses 1-38, wherein the fluid in the BFS vial comprises a vaccine, a drug, a medicament, or a component of any of the foregoing.

Clause 40. The pre-filled medical delivery assembly of any clause or example herein, in particular, any one of clauses 1-39, wherein the fluid seal comprises a foil, wax, paper, a section of the BFS vial, or any combination of the foregoing.

Clause 41. A pre-filled medical delivery assembly, comprising:
a blow-fill-seal (BFS) bottle defining a collapsible fluid chamber, a neck portion, an exterior flange formed on the neck portion, and a BFS seal at an end of the neck portion; and
a piercing connector defining a first end and a second end, the first end comprising an interior seat into which the exterior flange is axially mated, the second end comprising threads, and a piercing element disposed in an interior volume defined by the piercing connector such that a seating of the exterior flange in the interior seat causes the piercing element to puncture the BFS seal.

Clause 42. The pre-filled medical delivery assembly of any clause or example herein, in particular, clause 41, wherein the piercing element comprises a cross-shaped plastic projection that defines fluid passages in between the elements forming the cross-shape.

Clause 43. The pre-filled medical delivery assembly of any clause or example herein, in particular, any one of clause 41-42, wherein the piercing element comprises a conical plastic projection that comprises at least one fluid bore disposed therethrough.

Clause 44. The pre-filled medical delivery assembly of any clause or example herein, in particular, any one of clause 41-43, wherein the piercing element comprises a pointed metal tube.

Clause 45. The pre-filled medical delivery assembly of any clause or example herein, in particular, any one of clause 41-44, wherein the BFS bottle contains a fluid.

Clause 46. A connector for breaching a fluid seal of a blow-fill-seal (BFS) vial, the connector comprising:
an inlet port connecting to a first interior volume;
an outlet port connecting to a second interior volume in fluid communication with the first interior volume; and a piercing element having a least a piercing tip disposed within the first interior volume,
wherein the inlet port is constructed to receive insertion of a neck portion of the BFS vial therein.

Clause 47. A connector for breaching a fluid seal of a blow-fill-seal (BFS) vial, the connector comprising:
an inlet port connecting to a first interior volume;
an outlet port connecting to a second interior volume in fluid communication with the first interior volume; and means for piercing the fluid seal of the BFS vial,
wherein the inlet port is constructed to receive insertion of a neck portion of the BFS vial therein.

Clause 48. The connector of any clause or example herein, in particular, any one of clauses 46-47, wherein the inlet port and the outlet port are substantially coaxial and disposed at opposite longitudinal ends of the connector.

Clause 49. The connector of any clause or example herein, in particular, any one of clauses 46-48, wherein the connector comprises a longitudinally-extending nozzle that forms the outlet port.

Clause 50. The connector of any clause or example herein, in particular, clause 49, wherein the connector further comprises a longitudinally-extending annular flange that at least partially surrounds the nozzle.

Clause 51. The connector of any clause or example herein, in particular, clause 50, wherein a radially-inner surface of the annular flange, a radially-outer surface of the annular flange, or both comprise threads.

Clause 52. The connector of any clause or example herein, in particular, any one of clauses 50-51, wherein the annular flange, the nozzle, or both form a luer lock connection member.

Clause 53. The connector of any clause or example herein, in particular, any one of clauses 49-52, wherein a radially-outer surface of the nozzle comprises threads.

Clause 54. The connector of any clause or example herein, in particular, any one of clauses 49-53, wherein the nozzle forms a luer lock connection member.

Clause 55. The connector of any clause or example herein, in particular, any one of clauses 46-54, wherein the piercing element or the means for piercing defines at least a portion of a flow path from an interior of the neck portion to the second interior volume.

Clause 56. The connector of any clause or example herein, in particular, any one of clauses 46-55, wherein an interior sidewall of the connector that forms the first interior volume comprises a mating feature constructed to receive therein a laterally-protruding portion of the BFS vial so as to couple the BFS vial to the connector.

Clause 57. The connector of any clause or example herein, in particular, clause 56, wherein the mating feature comprises a scalloped portion of the interior sidewall.

Clause 58. The connector of any clause or example herein, in particular, any one of clauses 46-57, wherein the connector comprises a body portion that defines the inlet port, and the body portion has one or more anti-rotation coupling features configured to interact with flange portions of the BFS vial to inhibit rotation thereof with respect to the connector.

Clause 59. The connector of any clause or example herein, in particular, clause 58, wherein the anti-rotation coupling features comprises one or more longitudinally-extending slots through a sidewall of the body portion, one or more laterally-indented portions disposed around a circumference of the inlet port, or any combination of the foregoing.

Clause 60. The connector of any clause or example herein, in particular, any one of clauses 46-57, wherein the connector comprises means for inhibiting rotation of the BFS vial with respect to the connector.

Clause 61. The connector of any clause or example herein, in particular, any one of clauses 46-60, wherein the piercing element or the means for piercing comprises at least first and second crossmembers.

Clause 62. The connector of any clause or example herein, in particular, clause 61, wherein the at least first and second crossmembers are arranged to form a cross shape.

Clause 63. The connector of any clause or example herein, in particular, any one of clauses 61-62, wherein the second crossmembers are disposed on opposite sides of and extend orthogonal to the first crossmember, and/or the first crossmember extends further along a longitudinal direction than the second crossmembers such that a central portion of the first crossmember forms the piercing tip.

Clause 64. The connector of any clause or example herein, in particular, any one of clauses 61-63, wherein the first interior volume is defined at least in part by a chamber end wall of the connector, and/or the piercing element longitudinally extends from the chamber end wall toward the inlet port, and/or the chamber end wall comprises one or more openings fluidically connecting the first interior volume to the second interior volume.

Clause 65. The connector of any clause or example herein, in particular, clause 64, wherein the chamber end wall has a plurality of the openings, each opening corresponding to and connecting with a respective quadrant or region defined by the cross shape of the piercing element.

Clause 66. The connector of any clause or example herein, in particular, any one of clauses 61-65, wherein the first and second crossmembers of the piercing element are substantially solid.

Clause 67. The connector of any clause or example herein, in particular, any one of clauses 46-60, wherein the piercing element or the means for piercing comprises a conical or frustoconical member.

Clause 68. The connector of any clause or example herein, in particular, clause 67, wherein the conical or frustoconical member comprises one or more lumens longitudinally extending therethrough so as to fluidically connect the first interior volume to the second interior volume.

Clause 69. The connector of any clause or example herein, in particular, any one of clauses 67-68, wherein the first interior volume is defined at least in part by a chamber end wall of the connector, and/or the conical or frustoconical member longitudinally extends from the chamber end wall toward the inlet port.

Clause 70. The connector of any clause or example herein, in particular, any one of clauses 46-69, wherein the piercing element or the means for piercing is integrally formed with the connector.

Clause 71. The connector of any clause or example herein, in particular, any one of clauses 46-60, wherein the piercing element or the means for piercing comprises a hollow metal tube, and/or the piercing tip is formed by a beveled edge of the metal tube.

Clause 72. The connector of any clause or example herein, in particular, clause 71, wherein the connector comprises a longitudinally-extending nozzle that forms the outlet port, and the hollow metal tube is at least partially embedded within the nozzle.

### X. Rules of Interpretation

Any or all of the components disclosed herein can be formed of one or more plastics. In some embodiments, some components (e.g., the BFS vials) can be formed of a relatively soft polymer (e.g., having a Shore/Durometer “D” hardness of between 60 and 70), such as polyethylene (e.g., low density polyethylene (LDPE)), polypropylene, or any other polymer adaptable for use in a BFS manufacturing process. In some embodiments, some components (e.g., the connection assemblies, the administration assemblies, and/or needle caps or covers) can be formed, at least in part, of a relatively hard polymer (e.g., having a hardness greater than 80 on the Rockwell “R” scale), such as, but not limited to, polypropylene, polycarbonate, polybenzimidazole, acrylonitrile butadiene styrene (ABS), polystyrene, polyvinyl chloride, or the like. Other materials are also possible according to one or more contemplated embodiments.

Throughout the description herein and unless otherwise specified, the following terms may include and/or encompass the example meanings provided. These terms and illustrative example meanings are provided to clarify the language selected to describe embodiments both in the specification and in the appended claims, and accordingly, are not intended to be generally limiting. While not generally limiting and while not limiting for all described embodiments, in some embodiments, the terms are specifically limited to the example definitions and/or examples provided. Other terms are defined throughout the present description.

Numerous embodiments are described in this patent application, and are presented for illustrative purposes only. The described embodiments are not, and are not intended to be, limiting in any sense. The presently disclosed invention(s) are widely applicable to numerous embodiments, as is readily apparent from the disclosure. One of ordinary skill in the art will recognize that the disclosed invention(s) may be practiced with various modifications and alterations, such as structural, logical, software, and electrical modifications. Although particular features of the disclosed invention(s) may be described with reference to one or more particular embodiments and/or drawings, it should be understood that such features are not limited to usage in the one or more particular embodiments or drawings with reference to which they are described, unless expressly specified otherwise.

The present disclosure is neither a literal description of all embodiments of the invention nor a listing of features of the invention that must be present in all embodiments.

Neither the Title (set forth at the beginning of the first page of this patent application) nor the Abstract (set forth at the end of this patent application) is to be taken as limiting in any way as the scope of the disclosed invention(s).

While the term “vials” is utilized herein for convenience and ease of illustration, objects represented and/or described as “vials” may comprise various forms, configurations, and/or quantities of components. A BFS vials may comprise one or more BFS products that are formed and/or manufactured together or separately, for example, and/or may comprise one or more BFS modules, chambers, bottles, containers, and/or other fluid-retaining objects. The term “vials” does not convey any designation of shape or size. In some embodiments, a BFS assembly may comprise one or more vials. According to some embodiments a BFS assembly and/or a BFS vial may comprise one or more fluid chambers. In some embodiments, a plurality of BFS modules, components, vials, and/or chambers may be manufactured simultaneously from a single BFS mold. Each respective vial and/or chamber may be formed, for example, by different portions of a single BFS mold (e.g., two cooperative halves thereof). In some embodiments, BFS modules, components, vials, and/or chambers may be joined and/or coupled during manufacturing (e.g., via unformed and/or fused connecting parison) and/or after manufacturing/filling.

The term “product” means any machine, manufacture and/or composition of matter as contemplated by 35 U.S.C. § 101, unless expressly specified otherwise.

The terms “an embodiment”, “embodiment”, “embodiments”, “the embodiment”, “the embodiments”, “one or more embodiments”, “some embodiments”, “one embodiment” and the like mean “one or more (but not all) disclosed embodiments”, unless expressly specified otherwise.

A reference to “another embodiment” in describing an embodiment does not imply that the referenced embodiment is mutually exclusive with another embodiment (e.g., an embodiment described before the referenced embodiment), unless expressly specified otherwise.

The terms “a”, “an” and “the” mean “one or more”, unless expressly specified otherwise.

The term “plurality” means “two or more”, unless expressly specified otherwise.

The term “herein” means “in the present application, including anything which may be incorporated by reference”, unless expressly specified otherwise.

The phrase “at least one of”, when such phrase modifies a plurality of things (such as an enumerated list of things) means any combination of one or more of those things, unless expressly specified otherwise. For example, the phrase at least one of a widget, a car and a wheel means either (i) a widget, (ii) a car, (iii) a wheel, (iv) a widget and a car, (v) a widget and a wheel, (vi) a car and a wheel, or (vii) a widget, a car and a wheel.

The phrase “based on” does not mean “based only on”, unless expressly specified otherwise. In other words, the phrase “based on” describes both “based only on” and “based at least on”.

Where a limitation of a first claim would cover one of a feature as well as more than one of a feature (e.g., a limitation such as “at least one widget” covers one widget as well as more than one widget), and where in a second claim that depends on the first claim, the second claim uses a definite article “the” to refer to the limitation (e.g., “the widget”), this does not imply that the first claim covers only one of the feature, and this does not imply that the second claim covers only one of the feature (e.g., “the widget” can cover both one widget and more than one widget).

Each process (whether called a method, algorithm or otherwise) inherently includes one or more steps, and therefore all references to a “step” or “steps” of a process have an inherent antecedent basis in the mere recitation of the term ‘process’ or a like term. Accordingly, any reference in a claim to a ‘step’ or ‘steps’ of a process has sufficient antecedent basis.

When an ordinal number (such as “first”, “second”, “third” and so on) is used as an adjective before a term, that ordinal number is used (unless expressly specified otherwise) merely to indicate a particular feature, such as to distinguish that particular feature from another feature that is described by the same term or by a similar term. For example, a “first widget” may be so named merely to distinguish it from, e.g., a “second widget”. Thus, the mere usage of the ordinal numbers “first” and “second” before the term “widget” does not indicate any other relationship between the two widgets, and likewise does not indicate any other characteristics of either or both widgets. For example, the mere usage of the ordinal numbers “first” and “second” before the term “widget” (1) does not indicate that either widget comes before or after any other in order or location; (2) does not indicate that either widget occurs or acts before or after any other in time; and (3) does not indicate that either widget ranks above or below any other, as in importance or quality. In addition, the mere usage of ordinal numbers does not define a numerical limit to the features identified with the ordinal numbers. For example, the mere usage of the ordinal numbers “first” and “second” before the term “widget” does not indicate that there must be no more than two widgets.

When a single device or article is described herein, more than one device or article (whether or not they cooperate) may alternatively be used in place of the single device or article that is described. Accordingly, the functionality that is described as being possessed by a device may alternatively be possessed by more than one device or article (whether or not they cooperate).

Similarly, where more than one device or article is described herein (whether or not they cooperate), a single device or article may alternatively be used in place of the more than one device or article that is described. For example, a plurality of computer-based devices may be substituted with a single computer-based device. Accordingly, the various functionality that is described as being possessed by more than one device or article may alternatively be possessed by a single device or article.

The functionality and/or the features of a single device that is described may be alternatively embodied by one or more other devices which are described but are not explicitly described as having such functionality and/or features. Thus, other embodiments need not include the described device itself, but rather can include the one or more other devices which would, in those other embodiments, have such functionality/features.

Devices that are in communication with each other need not be in continuous communication with each other, unless expressly specified otherwise. On the contrary, such devices need only transmit to each other as necessary or desirable, and may actually refrain from exchanging data most of the time. For example, a machine in communication with another machine via the Internet may not transmit data to the other machine for weeks at a time. In addition, devices that are in communication with each other may communicate directly or indirectly through one or more intermediaries.

A description of an embodiment with several components or features does not imply that all or even any of such components and/or features are required. On the contrary, a variety of optional components are described to illustrate the wide variety of possible embodiments of the present invention(s). Unless otherwise specified explicitly, no component and/or feature is essential or required.

Further, although process steps, algorithms or the like may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the invention, and does not imply that the illustrated process is preferred.

Although a process may be described as including a plurality of steps, that does not indicate that all or even any of the steps are essential or required. Various other embodiments within the scope of the described invention(s) include other processes that omit some or all of the described steps. Unless otherwise specified explicitly, no step is essential or required.

Although a product may be described as including a plurality of components, aspects, qualities, characteristics and/or features, that does not indicate that all of the plurality are essential or required. Various other embodiments within the scope of the described invention(s) include other products that omit some or all of the described plurality.

An enumerated list of items (which may or may not be numbered) does not imply that any or all of the items are mutually exclusive, unless expressly specified otherwise. Likewise, an enumerated list of items (which may or may not be numbered) does not imply that any or all of the items are comprehensive of any category, unless expressly specified otherwise. For example, the enumerated list “a computer, a laptop, a PDA” does not imply that any or all of the three items of that list are mutually exclusive and does not imply that any or all of the three items of that list are comprehensive of any category.

Headings of sections provided in this patent application and the title of this patent application are for convenience only, and are not to be taken as limiting the disclosure in any way.

“Determining” something can be performed in a variety of manners and therefore the term “determining” (and like terms) includes calculating, computing, deriving, looking up (e.g., in a table, database or data structure), ascertaining and the like

The terms “including”, “comprising” and variations thereof mean “including but not limited to”, unless expressly specified otherwise. As used herein, “comprising” means “including,” and the singular forms “a” or “an” or “the” include plural references unless the context clearly dictates otherwise. The term “or” refers to a single element of stated alternative elements or a combination of two or more elements, unless the context clearly indicates otherwise

A description of an embodiment with several components or features does not imply that all or even any of such components and/or features are required. On the contrary, a variety of optional components are described to illustrate the wide variety of possible embodiments of the present invention(s). Unless otherwise specified explicitly, no component and/or feature is essential or required.

Further, although process steps, algorithms or the like may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the invention, and does not imply that the illustrated process is preferred.

The present disclosure provides, to one of ordinary skill in the art, an enabling description of several embodiments and/or inventions. Some of these embodiments and/or inventions may not be claimed in the present application, but may nevertheless be claimed in one or more continuing applications that claim the benefit of priority of the present application. Applicants intend to file additional applications to pursue patents for subject matter that has been disclosed and enabled but not claimed in the present application.

It will be understood that various modifications can be made to the embodiments of the present disclosure herein without departing from the scope thereof. Therefore, the above description should not be construed as limiting the disclosure, but merely as embodiments thereof. Those skilled in the art will envision other modifications within the scope of the invention as defined by the claims appended hereto.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented byway of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods, as known to those of ordinary skill in the art. When directly and explicitly distinguishing embodiments from discussed prior art, the embodiment numbers are not approximates unless the word “about” is recited. Whenever “substantially,” “approximately,” “about,” or similar language is explicitly used in combination with a specific value, variations up to and including ten percent (10%) of that value are intended, unless explicitly stated otherwise.

Directions and other relative references may be used to facilitate discussion of the drawings and principles herein, but are not intended to be limiting. For example, certain terms may be used such as “inner,” “outer,”, “upper,” “lower,” “top,” “bottom,” “interior,” “exterior,” “left,” right,” “front,” “back,” “rear,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part can become a “lower” part simply by turning the object over. Nevertheless, it is still the same part and the object remains the same.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### XI. Conclusion

Any of the features illustrated or described with respect to FIGS. 1A-7B and Clauses 1-72 can be combined with any other features illustrated or described with respect to FIGS. 1A-7B and Clauses 1-72 to provide systems, assemblies, modules, products, methods, and embodiments not otherwise illustrated or specifically described herein. For example, the BFS vial 150 of FIGS. 1C-1D can be used in place of the BFS vial 110 in any of FIGS. 2A-2C, 3A-3B, 6A-6D, 6F, 7A, and 7B, and/or can be used in conjunction with the piercing elements of any of FIGS. 2A-7B. Other combinations and variations are also possible according to one or more contemplated embodiments. All features described herein are independent of one another and, except where structurally impossible, can be used in combination with any other feature described herein.

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A pre-filled medical delivery assembly comprising:
(i) a blow-fill-seal (BFS) vial having first and second longitudinal ends, the BFS vial comprising:
at least one reservoir having a fluid therein; and
a longitudinally-extending neck portion having a fluid seal at the first longitudinal end of the BFS vial; and
(ii) a connector comprising:
an inlet port connecting to a first interior volume;
an outlet port connecting to a second interior volume in fluid communication with the first interior volume; and
a piercing element having a least a piercing tip disposed within the first interior volume,
wherein the neck portion of the BFS vial extends through the inlet port into the first interior volume such that at least part of the piercing element breaches and extends through the fluid seal.

2. The pre-filled medical delivery assembly of claim 1, wherein the inlet port and the outlet port are substantially coaxial and disposed at opposite longitudinal ends of the connector.

3. The pre-filled medical delivery assembly of claim 1, wherein the connector comprises a longitudinally-extending nozzle that forms the outlet port.

4. The pre-filled medical delivery assembly of claim 3, wherein the connector further comprises a longitudinally-extending annular flange that at least partially surrounds the nozzle.

5. The pre-filled medical delivery assembly of claim 4, wherein a radially-inner surface of the annular flange comprises threads.

6. The pre-filled medical delivery assembly of claim 5, wherein the annular flange, the nozzle, or both form a luer lock connection member.

7. The pre-filled medical delivery assembly of claim 3, wherein a radially-outer surface of the nozzle comprises threads.

8. The pre-filled medical delivery assembly of claim 7, wherein the nozzle forms a luer lock connection member.

9. The pre-filled medical delivery assembly of claim 1, wherein the piercing element defines at least a portion of a flow path from an interior of the neck portion to the second interior volume.

10. The pre-filled medical delivery assembly of claim 1, wherein an interior sidewall of the connector that forms the first interior volume comprises a mating feature, and the neck portion of the BFS vial comprises a laterally-protruding portion that is received in the mating feature so as to couple the BFS vial to the connector.

11. The pre-filled medical delivery assembly of claim 10, wherein the mating feature comprises a scalloped portion of the interior sidewall, and each laterally-protruding portion has a toroidal shape in a side view.

12. The pre-filled medical delivery assembly of claim 1, wherein:
the BFS vial further comprises flange portions disposed on opposite lateral sides of a body portion of the BFS vial and proximal to a junction of the body portion with the neck portion; and
the connector comprises a body portion that defines the inlet port, the body portion having one or more anti-rotation coupling features that interact with the flange portions of the BFS vial to inhibit rotation of the BFS vial with respect to the connector.

13. The pre-filled medical delivery assembly of claim 12, wherein the anti-rotation coupling features comprises one or more longitudinally-extending slots through a sidewall of the body portion, one or more laterally-indented portions disposed around a circumference of the inlet port, or any combination of the foregoing.

14. The pre-filled medical delivery assembly of claim 1, wherein the piercing element comprises at least first and second crossmembers.

15. The pre-filled medical delivery assembly of claim 14, wherein the at least first and second crossmembers are arranged to form a cross shape.

16. The pre-filled medical delivery assembly of claim 15, wherein the second crossmembers are disposed on opposite sides of and extend orthogonal to the first crossmember, the first crossmember extending further along a longitudinal direction than either of the second crossmembers such that a central portion of the first crossmember forms the piercing tip.

17. The pre-filled medical delivery assembly of claim 15, wherein the first interior volume is defined at least in part by a chamber end wall of the connector, the piercing element longitudinally extends from the chamber end wall toward the inlet port, and the chamber end wall comprises one or more openings fluidically connecting the first interior volume to the second interior volume.

18. The pre-filled medical delivery assembly of claim 17, wherein the chamber end wall has a plurality of the openings, each opening corresponding to and connecting with a respective quadrant defined by the cross shape of the piercing element.

19. The pre-filled medical delivery assembly of claim 14, wherein the piercing element is integrally formed with the connector, and the connector and the piercing element are formed of a plastic having a hardness greater than that of the BFS vial.

20. The pre-filled medical delivery assembly of claim 14, wherein the first and second crossmembers of the piercing element are substantially solid, such that the fluid from the at least one reservoir flows around the crossmembers en route to the second interior volume.

21. The pre-filled medical delivery assembly of claim 1, wherein the piercing element comprises a conical or frustoconical member.

22. The pre-filled medical delivery assembly of claim 21, wherein the conical or frustoconical member comprises one or more lumens longitudinally extending therethrough so as to fluidically connect the first interior volume to the second interior volume.

23. The pre-filled medical delivery assembly of claim 21, wherein the first interior volume is defined at least in part by a chamber end wall of the connector, and the conical or frustoconical member longitudinally extends from the chamber end wall toward the inlet port.

24. The pre-filled medical delivery assembly of claim 21, wherein the piercing element is integrally formed with the connector, and the connector and the piercing element are formed of a plastic having a hardness greater than that of the BFS vial.

25. The pre-filled medical delivery assembly of claim 1, wherein the piercing element comprises a hollow metal tube, and the piercing tip is formed by a beveled edge of the metal tube.

26. The pre-filled medical delivery assembly of claim 25, wherein the connector comprises a longitudinally-extending nozzle that forms the outlet port, and the hollow metal tube is at least partially embedded within the nozzle.

27. The pre-filled medical delivery assembly of claim 25, wherein the connector is formed of a plastic having a hardness greater than that of the BFS vial.

28. The pre-filled medical delivery assembly of claim 1, further comprising:
(iii) an administration assembly comprising:
a hub coupled to the outlet port of the connector; and
at least one administration conduit having an outlet at an end thereof longitudinally spaced from the hub and being in fluid communication with the second interior volume.

29. The pre-filled medical delivery assembly of claim 28, wherein the hub is coupled to the outlet port via a luer lock connection.

30. The pre-filled medical delivery assembly of claim 28, wherein at least part of the administration assembly is formed separate from and constructed to be coupled to the connector, or at least part of the administration assembly is integrally formed with the connector.

31. The pre-filled medical delivery assembly of claim 28, wherein the at least one administration conduit comprises a needle or cannula.

32. The pre-filled medical delivery assembly of claim 31, wherein the needle has a length in a range of 0.5 mm to 4 mm, inclusive, or in a range of 4 mm to 15 mm, inclusive, or in a range of 15 mm to 30 mm, inclusive.

33. The pre-filled medical delivery assembly of claim 28, wherein the outlet of the at least one administration conduit is formed as a nozzle configured to facilitate dispersion of the fluid from the BFS vial into a spray, or the outlet of the at least one administration conduit is formed as a nozzle configured to facilitate dispersion of the fluid from the BFS vial into one or more droplets.

34. The pre-filled medical delivery assembly of claim 1, further comprising (iii) an intravenous (IV) therapy device coupled to the outlet port of the connector.

35. The pre-filled medical delivery assembly of claim 34, wherein the IV therapy device is coupled to the outlet port via a luer lock connection.

36. The pre-filled medical delivery assembly of claim 1, wherein the fluid in the BFS vial comprises a vaccine, a drug, a medicament, or a component of any of the foregoing.

37. The pre-filled medical delivery assembly of claim 1, wherein the fluid seal comprises a foil, wax, paper, a section of the BFS vial, or any combination of the foregoing.
